(19) United States
(12) Patent Application Publication
(10) Pub. No.: US 2012/0244629 A1
(43) Pub. Date: Sep. 27, 2012

(54) METHOD FOR PREDICTING HIGH-GRADE VESICOURETERAL REFLUX IN CHILDREN WITH A FIRST FEBRILE URINARY TRACT INFECTION

(75) Inventors: Martin Chalumeau, Paris (FR); Sandrine Leroy, Paris (FR); Dominique Gendrel, Paris (FR)

(73) Assignee: ASSISTANCE PUBLIQUE - HOPITAUX DE PARIS, Paris (FR)

(21) Appl. No.: 13/383,929

(22) PCT Filed: Mar. 16, 2010
(86) PCT No.: PCT/FR2010/000223

§ 371 Date: Jun. 4, 2012

(30) Foreign Application Priority Data
Mar. 24, 2009 (FR) 09/01379

(51) Int. Cl. G01N 33/566

(52) U.S. Cl. 436/501

(57) ABSTRACT

A method for predicting a high-grade vesicoureteral reflux in a patient includes the steps of measuring the serum concentration of procalcitonin in a biological sample, assigning a procalcitonin value, looking for a dilation of one or two ureters shown on an echographic image of a kidney, assigning an echographic value, and combining the values by two mathematical equations with the definition of a threshold above which the patient is considered at risk for vesicoureteral reflux. A kit including a device for measuring the procalcitonin concentration and device for measuring the cross-section of the ureter is described.

### TECHNICAL FIELD OF THE INVENTION

This invention relates to a method for predicting vesicoureteral reflux.

This invention is particularly applicable in the medical field, and more specifically in the field of paediatrics, urology and detection of vesicoureteral reflux.

In the description below, the references between parentheses ( ) refer to the list of references provided after the examples.

### PRIOR ART

Febrile urinary tract infections are the most common invasive bacterial infections in humans (see Hoberman et al., J. Pediatr. 1993, “Prevalence of urinary tract infection in febrile infants.” (Ref. 1)). Indeed, 7% of girls and 2% of boys contract a febrile urinary infection before the age of 6 years (see Madrild, Acta Paediatr. 1998, “Incidence rate of first-time symptomatic urinary tract infection in children under 6 years of age.” (Ref. 2)).

Vesicoureteral reflux is discovered in 20% to 40% of infants during this event (see Jacobson, Acta Paediatr. 1999, “Vesico-ureteric reflux: occurrence and long-term risks.” (Ref. 3)).

Vesicoureteral reflux causes in particular an increase in the risk of recurrent infections, renal scarring, arterial hypertension and chronic renal failure (Ref. 3).

The current recommendation by learned paediatrics societies, in particular French (Guillot, Arch Pediatr. 1998, “Initial imaging in paediatric urinary tract infection. Arch Pediatr.” (Ref. 4)) and American (American Academy of Pediatrics, Committee on Quality Improvement, Subcommittee on Urinary Tract Infection. Practice parameter: the diagnosis, treatment and evaluation of the initial urinary tract infection in febrile infants and young children. Pediatrics 1999; 103: 843-52 (Ref. 5)) is to perform a cystography after a first febrile urinary infection. However, in 60 to 80% of cases, this cystography is normal and unnecessary, a posteriori.

Cystography is an irradiating examination for the gonads (0.8 mSv) (see Fotakis, Br. J. Radiol. 2003, “Radiation doses to paediatric patients up to 5 years of age undergoing micturating cystourethrography examinations and its dependence on patient age: a Monte Carlo study.” (Ref. 6)), involving a risk of iatrogenic urinary tract infection (Guignard, Lancet 1979, “Urinary infection after micturating cystography.” (Ref. 7)), and is painful (Hagglof, Acta Paedriatr. 1999, “Psychological reaction by children of various ages to hospital care and invasive procedures (Ref. 8)) and costly (Nicklasson, Acta Paedriatr. 1999, “Imaging studies after a first febrile urinary tract infection in young children.” (Ref. 9)) for the patient and social security.

Numerous studies have been conducted in order to find markers and decision rules making it possible to predict vesicoureteral reflux after a urinary tract infection and to avoid the practice of unnecessary cystography.

A first approach was that of using renal ultrasound as a diagnostic marker. Indeed, the presence of an abnormality in the renal ultrasound is considered by many paediatricians and radiologists to be a predictor of vesicoureteral reflux (Oualha M., Larakeb A., Grimprel E., Bensman A. Quand rechercher et comment prendre en charge un reflux vésico-urétéral en 2007? Journées Parisiennes de Pédiatrie. Flammarion Médecine-Sciences, Paris 2007; 1: 233-40).

In a non-systematic preliminary review of 15 studies published on the subject (see Chalumeau et al. “Procalcitonine et prédiction dureflux vésico-urétéral aprés une premiére infection urinaire fébrile chez l′enfant” Journée Parisienne de pédiatrie. Flammarion Médecine-Sciences, Paris 2005: 155-62. (Ref. 10)), it was shown that the results of studies analyzing the benefit of renal ultrasound in the prediction of vesicoureteral reflux were contradictory, in particular due to the diversity of the predictive ultrasound criteria used: pyelic dilation alone or combined with urethral dilation, kidney size, ultrasound as a whole.

In 2005, Westwood et al. conducted a systematic review of the literature and a meta-analysis of the complementary examinations (including renal ultrasound) to be performed after a urinary tract infection in children under 5 years of age (Westwood M. E., Whiting P. F., Cooper J., Watt I. S., Kleijnen J. Further investigation of confirmed urinary tract infection (UTI) in children under five years: a systematic review. BMC Pediatr. 2005; 5: 2. (Ref. 11)). They concluded that standard renal ultrasound is of minor benefit in predicting vesicoureteral reflux. However, these results were highly heterogeneous (defined in Cucherat M. Méta-analyse des essais thérapeutiques. Paris, France: Masson, 1997 (Ref. 12)), which made it difficult to interpret the results; the heterogeneity was not explored, and is assumed to be related in particular to the diversity of ultrasound criteria collected.

In addition, studies that appear to be major, such as that of Hoberman et al. (Hoberman A., Charron M., Hickey R. W., Baskin M., Kearney D. H., Wald E. R. Imaging studies after a first febrile urinary tract infection in young children. N. Engl. J. Med. 2003; 348: 195-202. (Ref. 13), were not included, without any clear justification. Therefore, the role of renal ultrasound in the prediction of vesicoureteral reflux is difficult to clearly establish and its precise benefit must be better defined and specified.

A second approach was the search for other markers for the detection of vesicoureteral reflux.

The documents of Leroy et al., “Procalcitonin as a predictor for vesico-ureteral reflux in children with urinary tract infection.” (June 2005, Pediatrics 2005; 115: e706-709) (Ref. 14) and Leroy et al., “Procalcitonin to reduce the number of unnecessary cystographies after a urinary tract infection in children: a European validation study.” (Journal of Pediatrics, January 2007; 150: 89-95) (Ref. 16) indicate that the serum concentration of procalcitonin can be considered to be a marker of vesicoureteral reflux. However, this marker has a low specificity, less than 44%.

These documents conclude that this marker alone does not make it possible to predict vesicoureteral reflux with enough specificity to avoid the practice of cystography.

Finally, a third approach involved the development of a decision rule (this term is defined in Wasson J. H., Sox H. C., Neff R. K., Goldman L. Clinical prediction rules. Applications and methodological standards. N. Engl. J. Med. 1985; 313: 793-9 (Ref. 15)) in order to detect vesicoureteral reflux after a first urinary tract infection.

The article of Oostenbrink et al., “Prediction of vesicoureteric reflux in childhood urinary tract infection: a multivariative approach (Acta Paediatr. 2000; 89: 806-10) (Ref. 17) describes a decision rule based on the study of clinical signs: urological family history, sex, age, biological characteristics: increase in C-reactive protein (abbreviated CRP in the rest of this document) and radiological characteristics: ultrasound observation of urinary tract dilation. According to this article, this rule would make it possible to obtain a sensitivity of 100% detection, but with a very low specificity of 38%.

The article of Leroy et al., March 2006, “Prediction of vesicoureteral reflux after a first febrile urinary tract infection in children: validation of a clinical decision rule.” (Archives of Disease in Childhood 2006; 91; 241-244) (Ref. 18), sought to test the reproducibility of the decision rule of Oostenbrink et al. in order to predict vesicoureteral reflux in children with a first febrile urinary tract infection. This article clearly shows that the aforementioned decision rule is insufficient because it has a very low reproducibility of results, with a failure to reproduce the 100% sensitivity and a reduction in specificity to 13% for the prediction of high-grade vesicoureteral reflux.

In the various approaches of the prior art, none made it possible to reduce the practice of cystography in order to detect a vesicoureteral reflux after a first urinary tract infection and which, in 60 to 80% of cases, is unnecessary, a posteriori.

There is therefore a real need to provide reliable markers and a less invasive prediction method than non-invasive cystography of said markers, making it possible to overcome the numerous disadvantages of the prior art, and in particular to obtain a fast, reliable and painless predictive diagnosis of vesicoureteral reflux, in order to detect it as early and as safely as possible and to reduce or even prevent the unnecessary practice of cystography.

### DESCRIPTION OF THE INVENTION

This invention is specifically intended to respond to this need and these disadvantages of the prior art by providing a method for predicting vesicoureteral reflux.

The method of the invention is a method for predicting high-grade vesicoureteral reflux in a patient.

The method of the invention includes the following steps:

a) measuring the serum concentration of procalcitonin in a biological sample of said patient,

b) assigning a procalcitonin value of 1 if the procalcitonin concentration is greater than or equal to an interval of values ranging from 0.4 ng/mL to 3 ng/mL, preferably ranging from 0.5 ng/mL to 2 ng/mL, preferably greater than or equal to 1 ng/ml in said sample, or assigning a procalcitonin value of 0 if the procalcitonin concentration is less than or equal to a concentration ranging from 0.4 ng/mL to 3 ng/mL, preferably from 0.5 ng/mL to 2 ng/mL, and preferably 1 ng/ml in said sample,

c) searching for dilation of one or both ureters shown on a standard renal ultrasound of said patient, assigning an ultrasound value of 1 if there is dilation of one or both ureters or an ultrasound value of 0 if there is no dilation,

d) adding said procalcitonin and ultrasound values obtained in steps b) and c), respectively, in which a sum greater than or equal to 1 indicates that the patient is considered to be at risk for vesicoureteral reflux.

In other words, in the method of the invention, in step b), a reference value is chosen in the interval of values defined above. This reference value can be, for example, between 0.4 ng/mL to 3 ng/mL, preferably between 0.5 ng/mL to 2 ng/mL, and preferably equal to 1 ng/ml. A procalcitonin value of 1 is assigned if the procalcitonin concentration measured in the sample in step a) is greater than or equal to said reference value. A procalcitonin value of 0 is assigned if the procalcitonin concentration is less than or equal to said reference value.

In this invention, a sum greater than or equal to 1 indicates that the patient is at high risk for vesicoureteral reflux, with an 86% sensitivity for predicting high-grade vesicoureteral reflux.

In this invention, a sum equal to 0 indicates that the patient is at low risk for high-grade vesicoureteral reflux, with less than 5% probability of overlooking high-grade vesicoureteral reflux.

This method has an 86% sensitivity and a 53% specificity for prediction of high-grade vesicoureteral reflux. A value equal to 0 indicates less than 5% probability of overlooking high-grade vesicoureteral reflux.

According to a second embodiment of the method of the invention, the method includes the following steps:

a) measuring the serum concentration of procalcitonin in a biological sample of said patient,

b) searching for dilation of one or both ureters shown on a standard renal ultrasound image of said patient, assigning an ultrasound value of 1 if there is dilation of one or both ureters or an ultrasound value of 0 if there is no dilation,

dbis) determining a reflux value Vr according to the following equation:

Vr=1/[1+exp(−a*urethral dilation−b*[(Pct/100)^{(c)}+d]−e)]

in which a, b, c, d and e are decimal numbers resulting or calculated by the logistic regression equation, which combines urethral dilation and procalcitonin converted into a first-degree fractional polynomial, and

in which each value of a, b, c, d and e is independently between −50 and 50, and a reflux value greater than a value between 0 and 1 indicates that the patient is at high risk for high-grade vesicoureteral reflux.

The values of a, b, c, d and e are calculated or result from the logistic regression equation combining the urethral dilation variable (considered to be binary, i.e. capable of taking the values 0 or 1) and the procalcitonin variable (considered to be continuous and converted into a first-degree fractional polynomial in order to satisfy the linearity hypothesis). (See Royton P., Sauerbrei W., Multivariable model-building Ed Wiley, 2008) (Ref. 19).

Each value of a, b, c, d and e can, for example, be chosen independently between −50 and 50, for example between −40 and 40, −30 and 30, −20 and 20, −10 and 10 and −5 and 5.

A reflux value greater than a value of between 0 and 1 indicates that the patient is at high risk for high-grade vesicoureteral reflux.

Preferably, the reflux value greater than 0.072 indicates that the patient is at high risk for high-grade vesicoureteral reflux with 86% sensitivity and 47% specificity.

Preferably, the reflux value below 0.072 indicates that the patient is a low risk for high-grade vesicoureteral reflux with less than 5% probability of overlooking a high-grade vesicoureteral reflux.

In addition, a reflux value resulting from the mathematical equation below a value of between 0 and 1, preferably 0.0072, indicates that the patient belongs to a low-risk group, with less than 5% probability of overlooking a high-grade vesicoureteral reflux.

Preferably, the values a, b, c, d and e are −1.5 for a, −0.1 for b, −0.5 for c, −4.9 for d and −1.5 for e.

The assignments of steps b) and c) and the summing of step d) can be performed automatically, for example by means of a software program based on the measured serum concentration of procalcitonin, the chosen reference value in the range of values defined above and the dilation of one of the two ureters or the non-dilation.

Vesicoureteral reflux can be classified according to grade. Grade I corresponds to reflux on a portion of the ureter. Grade II corresponds to urine reflux over the entire length of the ureter. Grade III corresponds to urine reflux over the entire length of the ureter with dilation of the ureter and pyelocaliceal cavities. Grade IV corresponds to urine reflux over the entire length of the ureter and in the pyelocaliceal cavities with marked dilation of the ureter and pyelocaliceal cavities. Grade V corresponds to urine reflux over the entire length of the ureter and in the pyelocaliceal cavities with dilation and a marked curvature of the ureter and the pyelocaliceal cavities according to the international classification (Lebowitz R. L., Olbing H., Parkkulainen K. V., Smellie J. M., Tamminen-Mobius T. E. International System of radiographic grading of vesicoureteric reflux. International Reflux Study in Children. Pediatr. Radiol. 1985; 15: 105-9. (Ref. 21)).

High-grade vesicoureteral reflux is reflux with a grade higher than or equal to III (see, for example, Hoberman et al., “Imaging studies after a first febrile urinary tract infection in young children.” NEJM 2003 (Ref. 4).

In this document, the term “patient” refers to any individual capable of having high-grade vesicoureteral reflux. More specifically, it can be, for example, a mammal, preferably a human being, for example an infant or child between the ages of 1 month and 11 years, an adolescent between the ages of 11 and 17 years, an adult between the ages of 18 and 99 years, and preferably an infant or child between the ages of 1 month and 4 years.

In this invention, the biological sample can be a blood, serum or plasma sample.

In this invention, the biological sample can come, for example, from a mammal, preferably a human being.

The biological sample can come, for example, from a healthy individual, i.e. not having any pathology, an individual with a disease or having clinical signs related to a disease, for example, fever, and/or an individual with a first febrile urinary tract infection.

Advantageously, the biological sample comes from an individual having a first febrile urinary tract infection.

It can, for example, be from an individual having a fever (rectal temperature greater than or equal to 38° C.) and bacteriuria.

The term “bacteriuria” refers to the presence of at least one bacterial colony in the individual's urine. Bacteriuria can be measured from a urine sample and the culturing of said sample. The urine sample can be obtained, for example, by a sterile perineal collection bag changed every 30 minutes, or by collecting the urine mid-stream, or by urethral catheter, or by suprapubic aspiration according to techniques well known to a person skilled in the art. Bacteriuria can be measured after 48 hours of culture of said urine sample, according to techniques well known to a person skilled in the art. The bacteriuria positivity thresholds are 10⁵ colonies forming a unit per mL (CFU/mL) for urine collected by a bag or mid-flow, 10⁴ CFU/mL for urine collected by urethral catheter, and 10¹ CFU/mL for urine collected by suprapubic aspiration (see, for example: Downs S. M. Technical report: urinary tract infections in febrile infants and young children. The Urinary Tract Subcommittee of the American Academy of Pediatrics Committee on Quality Improvement. Pediatrics. 1999; 103: e54 (Ref. 19)).

Advantageously, it is a blood sample of an individual with a febrile urinary tract infection and capable of having vesicoureteral reflux.

The biological sample can be collected at any time, for example, when the first clinical signs relating to a pathology appear, for example 1 hour after, 2 hours after, or up to 8 hours after a diagnosis of a urinary tract infection involving, for example in human beings, of a positive bacteriuria and a rectal temperature above 38° C.

The biological samples can be obtained according to techniques well known to a person skilled in the art. For example, for a blood sample, it can be obtained by means, for example, of a needle equipped with a syringe inserted into a vein in the forearm or an anterior elbow region of an individual. For example, a sample of 1 to 3 mL of blood obtained in an infant may be sufficient to implemented the method according to this invention.

Advantageously, the biological sample can be treated before the procalcitonin concentration is measured in order to inhibit the normal bactericidal properties, in particular present in the blood, and any antimicrobial agents, for example, by diluting the blood and adding inhibitors such as sodium polyanetholsulfonate (SPS) at a concentration of 0.025%.

Advantageously, the biological sample can correspond to a serum or plasma sample of an individual. Such a serum or plasma sample can be obtained simply by a person skilled in the art by centrifugation of a blood sample and recovery of the supernatant.

In this document, the term “procalcitonin” refers to the precursor protein of calcitonin with 116 single-stranded amino acids described in the document U.S. Pat. No. 6,905,687, and commonly referred to as ProCT or PCT. In the event of an infection, the procalcitonin is expressed ubiquitously by tissues other than neuroendocrine thyroid tissue, but the exact nature of the cells responsible for its synthesis and secretion in the blood is still unknown and debated. Experiments conducted in animals and humans suggest that procalcitonin is involved in the inflammatory reaction, without its role being clearly established at present.

The measurement of the procalcitonin concentration in a biological sample can be performed by all standard methods known to a person skilled in the art. For example, a person skilled in the art can use quantitative immunological techniques using antibodies or fragments of antibodies that specifically bind procalcitonin, for example the ELISA technique or the techniques described in PCT application WO 97 20213. It can be determined conventionally by biological laboratory analyses.

For example, the measurement of the procalcitonin concentration can be performed by immunoassay, an immunoluminometric technique or an immunochromatographic technique.

For example, the measurement of procalcitonin by immunoassay can be performed by placing the sample in contact with an antibody that specifically binds procalcitonin according to the method described in Michael Meisner “Procalcitonin, a new, innovative infection parameter. Biochemical and clinical aspects”, 3rd revised and expanded edition, Stuttgart, Germany, Thieme, 2000 (Ref. 72)). The determination of the procalcitonin concentration by means of an antibody that specifically binds procalcitonin can be performed according to techniques well known to a person skilled in the art, such as, for example, quantitative immunological techniques such as the ELISA technique, techniques described in document WO 97 20213 and the methods implemented in the LUMItest® assemblies also referred to as B.R.A.H.M.S. PCT LIA, B.R.A.H.M.S. PCT KRYPTOR® and LIAISON B.R.A.H.M.S. PCT available from B.R.A.H.M.S. AG (Berlin, Germany).

The antibodies used can be monoclonal or polyclonal antibodies or fragments of same. For example, the antibodies can be antibodies marked directly or indirectly by means of a second antibody. The antibodies can be marked, for example, by an enzyme such as peroxidase, alkaline phosphatase and β-galactosidase, by a luminescent reagent such as flurescein, rhodamine, cyanine or by means of a second biotinylated antibody.

The monoclonal antibodies can, for example, be antibodies described in documents U.S. Pat. No. 6,451,311, U.S. Pat. No. 5,330,909 and U.S. Pat. No. 6,133,427, the antibodies available at ABCAM under references “ab14813”, “ab11498”, “ab11494”, “ab14817”, “ab14816” and “ab24454”, the antibody available at CHEMICON under reference “MAB3490” and the antibodies available at GeneTex®, Inc. under references “GTX14813”, “GTX11498”, “GTX11494”, “GTX14817” and “GTX14816”.

The antibody fragments can be chosen from the group including, for example, the fragments Fab, F(ab′)2, FV and sFv.

Advantageously, the measurement of the procalcitonin concentration can be performed by a rapid semi-quantitative immunochromatographic test. It is an immunochromatographic test using a mouse anti-katakalcin monoclonal antibody conjugated with a colloidal tracer and a lamb anti-katakalcin polyclonal antibody. The test requires an incubation period of 30 minutes, without any preliminary calibration required. It is performed on a serum or plasma sample, obtained after centrifugation and separation of the total blood sample taken from the patient. When the serum or plasma sample of the patient is applied, the tracer binds to the procalcitonin of the sample and forms antibody-antigen complexes. These migrate by capillary action through the test system, and the antibody-antigen complexes are fixed by the anti-calcitonin antibodies, forming sandwich complexes. At a procalcitonin concentration of ≧0.5 ng/ml, the sandwich complex is displayed in the form of a reddish band. The intensity of the colour of the band is directly proportional to the procalcitonin concentration of the sample, according to the following intervals: <0.5 ng/ml, 0.5-2 ng/ml, 2-10 ng/ml, and a 10 ng/ml.

The detailed protocol of the semi-quantitative assay of serum procalcitonin is presented in Chapter 6 (pages 176-183) of the book “Procalcitonin, a new, innovative infection parameter. Biochemical and clinical aspects. “Michael Meisner, 3rd revised and expanded edition, 2000 Thieme ((Ref. 72)).

According to the invention, the search for dilation can be performed on a standard ultrasound image.

According to the invention, the standard ultrasound image can be obtained by any technique and/or any means known to a person skilled in the art. For example, the ultrasound image can be in the form of a photograph, a print or in the form of an image on a medical imaging screen obtained from an ultrasound imaging device. The ultrasound image device can be any device known to a person skilled in the art, for example, it can be an ultrasound, for example Hitashi Vision 900, 8500, 6500, 5500, Siemens Acuson Antares System, PHILIPS HD1 5000.

The dilation of one or both ureters is observed, for example, when the cross-section of the ureter is visible on the ultrasound image. For example, the dilation can be observed when the cross-section of the ureter is greater than 1 mm in a transverse cross-section on the ureter visible on the ultrasound image.

According to another aspect, the invention also relates to a kit including:

(1) at least one means for measuring the procalcitonin concentration in a biological sample of an individual, and

(2) means for measuring the cross-section of a ureter of an individual.

Means for measuring the procalcitonin concentration can, for example, be a device for implementing one of the techniques for measuring the concentration as described above, and a reagent used to implement one of the techniques for measuring the concentration as described above.

For example, they can be reagents for implementing a technique for measuring the procalcitonin concentration, for example measurement techniques chosen from the group including an immunoassay, a Lumitest®-PCT immunoluminometric technique, or a PCT®-Q rapid semi-quantitative immunochromatographic test (BRAHMS AG, Hennigsdorf).

Means for measuring the concentration of procalcitonin can also be a rapid semi-quantitative immunochromatographic test, for example a PCT®-Q rapid semi-quantitative immunochromatographic test (BRAHMS AG, Hennigsdorf).

Means for measuring the cross-section of a ureter can include a renal ultrasound, with the production of an image of one or both ureter(s) according to a transverse cross-section, as described above. The ultrasound imaging device can be any device known to a person skilled in the art, for example, it can be an ultrasound, for example Hitashi Vision, 900, 8500, 6500, 5500, Siemens Acuson Antares System, or PHILIPS HDI 5000. The measurement of the dilation can be performed, for example, directly on the image obtained by means of a metric ruler, and/or during acquisition of the ultrasound image by the ultrasound imaging device software.

Other advantages may become apparent to a person skilled in the art when reading the following examples, illustrated by the appended figures, provided for illustrative purposes.

### BRIEF DESCRIPTION OF THE FIGURES

FIG. 1 shows a diagram retracing the identification and inclusion of studies in the meta-analysis of the predictive capabilities of renal ultrasound for vesicoureteral reflux.

FIG. 2 is a graph showing a summary of sensitivity curves as a function of (1—specificity) the diagnostic power of the renal ultrasound for vesicoureteral reflux (see example 1) (“summary receiver operator characteristic” (SROC)) or summaries of ROC curves (see Moses L., Littenberg B., Shapiro D. Combining independent studies of a diagnostic test into a summary ROC curve: data-analytical approaches and some additional consideration. Stat Med 1993; 12: 1293-316 (Ref. 100)) for the renal ultrasound, all ultrasound criteria combined, for the prediction of vesicoureteral reflux of any grade. The x-axis shows (1—specificity) and the y-axis shows the sensitivity. These terms “sensitivity” and “specificity” are defined in Huguier M., Flahault A. Biostatistiques au quotidien. Paris, France: Elsevier, 2003 (Ref. 101).

FIG. 3 is a graph showing the ROC curves (Falissard. Comprendre et utiliser les statistiques dans les sciences de la vie. 2005. 3^{rd} edition. Ed Masson. Paris, France (Ref. 102)) of CRP and procalcitonin for predicting high-grade vesicoureteral reflux. The x-axis shows (1—specificity) and the y-axis shows the sensitivity.

### EXAMPLES

### Example 1

### Review of Different Criteria of the Renal Ultrasound as a Marker of Vesicoureteral Reflux

This example shows a systematic review and a meta-analysis of the predictive capabilities of the different criteria of the standard renal ultrasound for vesicoureteral reflux.

### A) Methods

### General Description

A systematic review of the literature followed by a meta-analysis of the studies evaluating the renal ultrasound as a predictor of vesicoureteral reflux during a urinary tract infection in children was performed.

### Identification of the Studies

The publications were identified by a request in the databases MedLine, Cochrane central and Embase with the keywords vesico ureteral reflux*, ultrasonography*, and infant or adolescent or child (as medical subject heading terms and as words contained in the text) for the period between January 1985 and September 2006. A manual search of the lists of references, of the first 60 references found by the Internet link “related articles” of MedLine and of the first 60 references of the list of publications of the first and last authors of each article identified was also performed. No language restriction was applied, and articles published in Spanish, German and Italian were translated.

### Inclusion Criteria of the Studies

All of the prospective or retrospective cohort studies were included with the following inclusion criteria:
article studying renal ultrasound as a predictor of vesicoureteral reflux;
article including enough data to complete the contingency table;
at least 20 patients included (arbitrary threshold (Westwood M. E., Whiting P. F., Cooper J., Watt I. S., Kleijnen J. Further investigation of confirmed urinary tract infection (UTI) in children under five years: a systematic review. BMC Pediatr 2005; 5:2.);
patients without known uropathy at the time of radiological evaluation;
patients aged 0 to 18 years;
patients with:
a first urinary tract infection (defined by a positive bacteriuria, regardless of the collection mode); or
a urinary tract infection (defined by positive bacteriuria regardless of the urinary collection mode, regardless of the number of previous episodes), with an ultrasound and cystography being systematically performed;
or for which a renal ultrasound and cystography were performed with a maximum six-month interval, if the reason for these examinations was a urinary tract infection for more than 50% of the patients.

The studies with more than 50% of the data missing, or relating to ultrasound with a contrast product, or including only patients having antenatal hydronephrosis were not included.

Randomized controlled studies on the treatment of urinary tract infection (in which the patients very likely underwent a renal ultrasound and cystography given the current recommendations on imaging examinations after a urinary tract infection) were not taken into account because these tests have, as a common point, including only children with a normal renal ultrasonography.

### Identification and Extraction of Data

The inventors conducted the electronic and manual search in order to potentially eligible identify articles based on their summaries. The final list was reviewed by two other readers independently.

The inventors decided on the inclusion of eligible studies and extracted the data from each article independently. The following data was collected: publication year, study location, patient characteristics, sample size, imaging technique used to diagnose vesicoureteral reflux, and ultrasound criteria. The latter were (if the information was present): pyelic dilation, dilation of the pelvis and/or calices, ureter dilation, dilation of the urinary tract when the study did not specify which part of the urinary tract was dilated, thickening of the pelvic wall, thickening of the ureter wall, thickening of the wall of the pelvis and/or of the ureter, renal size. When the study did not provide any precision on the criteria taken into account, the study was classified as an unspecified ultrasound anomaly. The data was collected in a standardized Excel file so as to later produce contingency tables.

The extraction of data was validated by the inventors independently on a sample of ten articles selected at random. If two studies by the same group of authors might have partially included the same patients, the inventors contacted the authors to clarify this point and only the study including the most recent data was included.

### Statistical Analyses

The predicted variable was vesicoureteral reflux. When the data was available in the studies, the reflux was considered as follows: high-grade vesicoureteral reflux (≧3), against the absence of vesicoureteral reflux or reflux of grade 1 or 2, as described in Hoberman et al. NEJM 2003 “Imaging studies after a first febrile urinary tract infection in young children (Ref. 4).

First, the sensitivities, specificities, positive and negative likelihood ratios and diagnostic odds ratio were calculated for the prediction of vesicoureteral reflux of any grade, with the confidence interval (CI) of 95% with a DerSimonian and Laird's mixed-effect model (DerSimonian R. L., N. Meta-analysis in clinical trials. Control Clin. Trials 1986; 7: 177-88. (Ref. 23)). The definition of all of these terms (sensitivity, specificity, positive and negative likelihood ratios, odds ratio and confidence interval) is presented in Huguier M., Flahault A. Biostatistiques au quotidien. Paris, France: Elsevier, 2003 (Ref. 101), and Bouyer J., Hemon D., Cordier S., Derrienic F., Stucker I., Stengel B., Clavel J. Epidémiologie. Principes et méthodes. Paris: INSEM ed., 1993 (Ref. 103) for their standard use in epidemiology. Their use in the context of meta-analysis is specified in Glas A. L., Prins M. H., Bonsel G. J., Bossuyt P. M. The diagnostic odds ratio: a single indicator of test performance. J. Clin. Epidemiol. 2003; 56: 1129-35 (Ref. 104).

These indices were calculated, first, for each ultrasound criteria (including unspecified abnormal ultrasounds), then for the sub-group of studies that studied urinary tract dilation (whether or not the type of dilation was specified), and finally for all of the studies. In the event of a zero value in the contingency table, the value 0.5 was added in each cell of the table (according to the protocol described in Deeks J. J., Higgins J. P. T., Altman D. G., editors. Analysing and presenting results. In: Alderson P, Green S., Higgins J., editors. Cochrane Reviewers' Handbook 4.2.2 [updated November 2004]; Section 8. (Ref. 24). For the six studies having provided their results for each renal unit (and not for each patient) (see Dura Trave T., Gonzalez Montera R., Juste Ruys M., Gonzales de Dios F., Carratala Marco M., Moya Benavent M., Verdu Rico J., Caballero Calpena O. Utilidad de la gammagrafia rénal en la valoracion de la primera infeccion febrii en la edad pediatrica. An Esp Pediatr 1997; 47: 378-82. (Ref. 25); Evans E. D., Meyer J. S., Harty M. P., Bellah R. D. Assessment of increase in renal pelvic size on post-void sonography as a predictor of vesicoureteral reflux. Pediatr. Radiol. 1999; 29: 291-4. (Ref. 26); Kenney I. J., Negus A. S., Miller F. N. Is sonographically demonstrated mild distal ureteric dilatation predictive of vesicoureteric reflux as seen on micturating cystourethrography? Pediatr. Radiol. 2002; 32: 175-8. (Ref. 27); Muensterer O. J. Comprehensive ultrasound versus voiding cysturethrography in the diagnosis of vesicoureteral reflux. Eur. J. Pediatr. 2002; 161: 435-7. (Ref. 28); Peratoner. L., Pennesi M., Bordugo A., Melega R., Sorce P., Travan L., Minisini S., Zennaro F., Da Ronch L. Kidney length and scarring in children with urinary tract infection: importance of ultrasound scans. Abdom. Imaging 2005; 30: 780-5. (Ref. 29); Tsai Y. C., Hsu C. Y., Lin G. J., Wang C. J., Cheng C. H., Huang Y. H., Yen M. H., Hsia S. H., Yan D. C. Vesicoureteral reflux in hospitalized children with urinary tract infection: the clinical value of pelvic ectasia on renal ultrasound, inflammatory responses and demographic data. Chang Gung Med. J. 2004; 27: 436-42. (Ref. 30)), the numbers were not modified (by not dividing them by two in particular) for the calculation of overall indices.

Secondly, for each analysis, a heterogeneity search was conducted by observing graphs (“forest plots” or in-line graphics (Cucherat M. Méta-analyse des essais thérapeutiques. Paris, France: Masson, 1997. (Ref. 12)) of the results for the diagnostic odds ratio, then by the Q test (Higgins J. P., Thompson S. G., Deeks J. J., Altman D. G. Measuring inconsistency in meta-analyses. BMJ 2003; 327: 557-60. (Ref. 105)) and the calculation of the statistic I² (Ref. 105). A degree of significance <0.05 of the Q test or a statistic I2>50% were considered to indicate significant heterogeneity (see Egger M., Smith G. D., Altman D. G. Systematic reviews in health care: meta-analysis in context. London, United Kingdom: BMJ Publishing Group, 2001. (Ref. 31)). In the event of divergence, the result of the Q test was preferred because its greater capability for detecting heterogeneity was demonstrated (as described in Lijmer J. G., Bossuyt P. M., Heisterkamp S. H. Exploring sources of heterogeneity in systematic reviews of diagnostic tests. Stat. Med. 2002; 21: 1525-37. (Ref. 32)).

Thirdly, the SROC curves for each ultrasound criteria were constructed, by means of a regression model of Moses L., Littenberg B., Shapiro D. Combining independent studies of a diagnostic test into a summary ROC curve: data-analytical approaches and some additional consideration. Stat Med 1993; 12: 1293-316. (Ref. 33). In this method, the logarithmic value of the true positives and false positives of each study was used to estimate the parameters of the regression model. Then, the data was re-transformed by the exponential function so as to be represented in the SROC space. The SROC curve was then traced, showing the relationship between sensitivity and the number of false positives (1—specificity). The observation of these curves made it possible to visually demonstrate a threshold effect (explaining the heterogeneity): the curve then took the form of a “shoulder arm” or “elbow” (Zamoura J, Abraira V, Muriel A, Khan K, Coomarasamy A. Meta-Disc: a software for meta-analysis of test accuracy data. BMC Med. Res. Methodol. 2006; 6: 1-12. (Ref. 34)).

Fourthly, a threshold effect was statistically researched. For this, the Spearman correlation coefficient for the SROC curve as well as its degree of significance was calculated.

Fifthly, by a so-called “univariate” meta-regression according to the Littenberg and Moses method, the effect of the different possible sources of heterogeneity between the studies (other than the threshold effect) on the relationship between vesicoureteral reflux of any grade and the different ultrasound criteria was studied (Ref. 33). This type of model is said to be “univariate” because one model per co-variable, a possible source of heterogeneity, is constructed (and not a model including all of the co-variables together as in a conventional meta-regression). This type of analysis was performed only when the number of studies included in the analysis was greater than or equal to ten due to the risk of over-parameterization of the model (see Ref. 31). The co-variables included in the model were:
the type of study: prospective or retrospective;
the amount of data missing: <20% or k 20%;
the presence of a blind measurement when performing the cystography;
the type of population:
patients with a first febrile urinary tract infection;
patients with a febrile urinary tract infection (of which the number was not specified); or patients for whom a cystography and a renal ultrasound were performed for unspecified reasons;
the ultrasound criteria for the model concerning the analysis, all ultrasound criteria combined.

Seventhly, the prediction of high-grade vesico-ureteral reflux by the renal ultrasound according to the analysis plan was studied.

Finally, a publication bias was researched, first graphically by producing a funnel plot graph, (Ref. 12), then statistically by the Egger regression test (Egger M., Davey Smith G., Schneider M., Minder C. Bias in meta-analysis detected by a simple, graphical test. BMJ 1997; 315: 629-34. (Ref. 36)) for the detection of vesicoureteral reflux of any grade.

MetaDisc (registered trademark) software (Creative Commons Attribution, Birmingham, UK) (Zamoura J., Abraira V., Muriel A., Khan K., Coomarasamy A. Meta-Disc: a software for meta-analysis of test accuracy data. BMC Med. Res. Methodol. 2006; 6: 1-12 (30) and Stata/SE 8 (registered trademark) software (Statacorp, College Station, Tex., USA) were used for the statistical analysis.

### Results

### Characteristics of the Studies

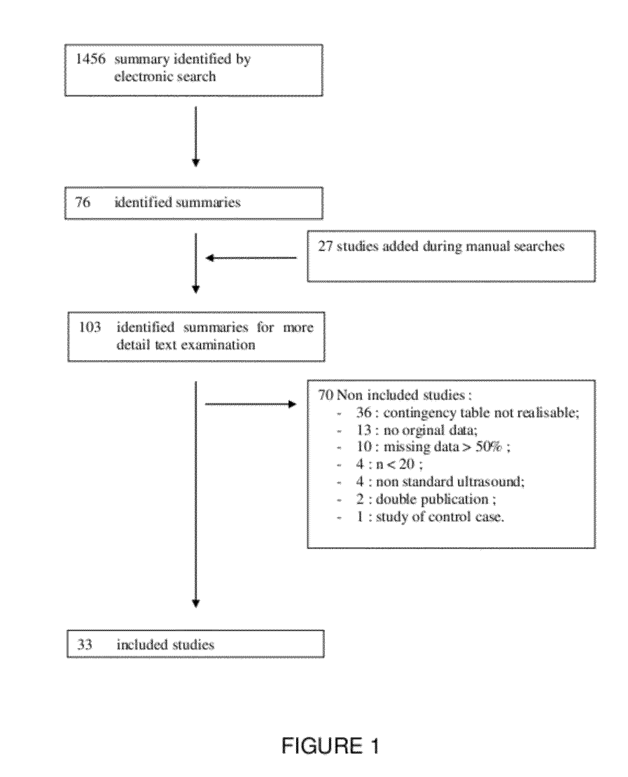

The electronic search on Medline, Cochrane Central and Embase identified 1456 summaries, of which 76 were considered to be potentially eligible (FIG. 1). The manual search found 27 additional studies, bringing the total to 103 potentially eligible articles. After reading the entire text, 70 studies were excluded: 36 studies were excluded because the contingency table could not be produced (unreported data, cystography performed only in the case of a positive ultrasound), 13 articles did not provide original data but were reviews of the literature or a perspective, ten studies had more than 50% missing data, four studies related to a patient group of less than 20, four studies related to an ultrasound technique other than standard ultrasound, two articles constituted double publications, and one study was a control case. In all, 33 studies were included: Hoberman A., Charron M., Hickey R. W., Baskin M., Kearney D. H., Wald E. R. Imaging studies after a first febrile urinary tract infection in young children. N. Engl. J. Med. 2003; 348: 195-202. (Ref. 13); (Ref. 23-28), Almeida H. N., Ribeiro M., Colarinha J., Santos J. F., Rosa F. C. Imaging methods in the study of urinary tract infections in children. Acta Med. Port. 1994; 7: 15-20. (Ref. 38); Alon U., Pery M., Davidai G., Berant M. Ultrasonography in the radiologic evaluation of children with urinary tract infection. Pediatrics 1986; 78: 58-64. (Ref. 39); Alon U.S., Ganaphthy S. Should renal ultrasonography be done routinely in children with first urinary tract infection? Clin. Pediatr. (Phila.) 1999; 38: 21-5. (Ref. 40); Baronciani D., Bonora G., Andreola A., Gambie M., Nedbal M., dell'Agnola C. A. Ruolo dell'ecografia nell'iter diagnostico delle infezioni delle vie urinarie. Riv. Ital. Ped. 1986; 12: 214-20. (Ref. 41); Boudailliez B., McMahon Y., Grumbach Y., Baratte B., Caraco M. H., Piussan C. Place de l′échographie dans l′investigation: une premiére infection urinaire chez l′enfant. Arch. Pediatr. 1989; 46: 113-6. (Ref. 42); Calisti A., Perrotta M. L., Oriolo L., Ingianna D., Sciortino R. Diagnostic workup of urinary tract infections within the first 24 months of life, in the era of prenatal diagnosis. The contribution of different imaging techniques to clinical management. Minerva Pediatr. 2005; 57: 269-73. (Ref. 43); Cleper R., Krause I., Eisenstein B., Davidovits M. Prevalence of vesicoureteral reflux in neonatal urinary tract infection. Clin. Pediatr. (Phila.) 2004; 43: 619-25. (Ref. 44); Davey M. S., Zerin J. M., Reilly C., Ambrosius W. T. Mild renal pelvic dilatation is not predictive of vesicoureteral reflux in children. Pediatr. Radiol. 1997; 27: 908-11. (Ref. 45); David C., Dacher J. N., Monroc M., Eurin D., Le Dosseur P. Retrograde cystography after a first episode of acute pyelonephritis in the child and adolescent. J. Radiol. 1998; 79: 133-7. (Ref. 46); DiPietro M. A., Blane C. E., Zerin J. M. Vesicoureteral reflux in older children: concordance of US and voiding cystourethrographic findings. Radiology 1997; 205: 821-2. (Ref. 47); Foresman W. H., Hulbert W. C., Rabinowitz R. Does urinary tract ultrasonography at hospitalization for acute pyelonephritis predict vesicoureteral reflux? J. Urol. 2001; 165: 2232-4. (Ref. 48); Gelfand M. J., Barr L. L., Abunku O. The initial renal ultrasound examination in children with urinary tract infection: the prevalence of dilated uropathy has decreased. Pediatr. Radiol. 2000; 30: 665-70. (Ref. 49); Goldman M., Lahat E., Strauss S., Reisler G., Livne A., Gordin L., Aladjem M. Imaging after urinary tract infection in male neonates. Pediatrics 2000; 105: 1232-5. (Ref. 50); Honkinen O., Ruuskanen O., Rikalainen H., Makinen E. O., Valimaki I.

Ultrasonography as a screening procedure in children with urinary tract infection. Pediatr. Infect. Dis. J. 1986; 5: 633-5. (Ref. 51); Leroy S., Adamsbaum C., Marc E., Moulin F., Raymond J., Gendrel D., Breart G., Chalumeau M. Procalcitonin as a predictor of vesicoureteral reflux in children with a first febrile urinary tract infection. Pediatrics 2005; 115: e706-9. (Ref. 52); Leroy S., Romanello C., Galetto-Lacour A., Smolkin V., Korczowski B., Rodrigo C., Tuerlinckx D., Gajdos V., Moulin F., Contardo M., Gervaix A., Haievy R., Duhl B., Prat C., Borght T. V., Foix-l'Helias L., Dubos F., Gendrel D., Breart G., Chalumeau M., Marc E., Adamsbaum C. “Procalcitonin to reduce the number of unnecessary cystographies in children with a urinary tract infection: a European validation study”. J. Pediatr. 2007; 150: 89-95, (Ref. 53); Mage K., Zoppardo P., Cohen R., Reinert P., Ponet M. Imaging and the first urinary infection in children. Respective role of each test during the initial evaluation a propos of 122 cases. J. Radiol. 1989; 70: 279-83, (Ref. 54); Mahant S., Friedman J., MacArthur C. Renal ultrasound findings and vesicoureteral reflux in children hospitalised with urinary tract infection. Arch. Dis. Child. 2002; 86: 419-20. (Ref. 55); Morin D., Veyrac C., Kotzki P. O., Lopez C., Dalla Vale F., Durand M. F., Astruc J., Dumas R. Comparison of ultrasound and dimercaptosuccinic acid scintigraphy changes in acute pyelonephritis. Pediatr. Nephrol. 1999; 13: 219-22. (Ref. 56); Oostenbrink R., van der Heijden A. J., Moons K. G., Moll H. A. Prediction of vesico-ureteric reflux in childhood urinary tract infection: a multivariate approach. Acta Paediatr. 2000; 89: 806-10. (Ref. 57); Smellie J. M., Rigden S. P., Prescod N. P. Urinary tract infection: a comparison of four methods of investigation. Arch. Dis. Child. 1995; 72. (Ref. 58); Tan. S M., Chee T., Tan K. P., Cheng H. K., Ooi B. C. Role of renal ultrasonography (RUS) and micturating cystourethrogram (MCU) in the assessment of vesico-ureteric reflux (VUR) in children and infants with urinary tract infection (UTI). Singapore Med. J. 1988; 29: 150-2. (Ref. 59); Taskinen S., Ronnholm K. Post-pyelonephritic renal scars are not associated with vesicoureteral reflux in children. J. Urol. 2005; 173: 1345-8. (Ref. 60); Yen C. W., Chen D. H. Urinary tract infection in children. J. Microbiol. Immunol. Infect. 1999; 32: 199-205. (Ref. 61); Zamir G., Sakran W., Horowitz Y., Koren A., Miron D. Urinary tract infection: is there a need for routine renal ultrasonography? Arch. Dis. Child. 2004; 89: 466-8. (Ref. 62)).

All of the studies were cohort studies. Seven prospective studies (Ref. 35, 24, 36, 41, 48, 51 and 60), 19 studies included patients consecutively (Ref. 35, 25, 26, 28, 37, 38, 42-48, 50, 51, 53, 55, 59 and 60), six studies indicated that the cystographic data was collected blindly from the renal ultrasound results (Ref. 37, 41, 43, 48, 50 and 51).

For five studies, contact had been lost with more than 20% of the patients before the cystography was performed (Ref. 23, 39, 46, 57 and 59). The data was analyzed per renal unit in six studies (Ref. 23-28) and per patient in the 27 other studies.

Vesicoureteral reflux was diagnosed and graded by a conventional radiological cystography in 28 articles (Ref. 35, 23-28, 38-46, 48, 50 and 52-61) and by a radiological or isotopic cystography in 5 studies (Ref. 36, 37, 47, 49 and 51).

Eighteen studies included patients with a first urinary tract infection (Ref. 35, 23, 28, 38, 40-42, 44, 48, 50-55 and 58-60). In 11 studies, the patients had a urinary tract infection (regardless of the number of similar antecedents, which was not specified) (Ref. 26, 27, 38, 39, 45-47, 49, 56, 57 and 61).

For four other studies, the patients were included if they had undergone a renal ultrasound and cystography within a fixed maximum time interval (ranging from one day to six months according to the article) regardless of the indication for the examination (Ref. 24, 26, 36 and 43). The analysis of data for the prediction of high-grade reflux was possible in ten studies (Ref. 35, 26, 28, 37, 39, 46, 50, 51, 53 and 60). The methodological quality of the studies is reported in Table 1. It appears to be highly variable and the studies were of poor methodological quality on the whole.

### Characteristics of the Patients

All of the studies included represented 5474 patients combined. Contact was lost with 420 patients (8%) before the cystography was performed, and the analysis concerning vesicoureteral reflux of any grade was therefore conducted on the data of 5054 patients (92%). One thousand, four hundred and seventy patients (27%) had vesicoureteral reflux. This reflux was high grade (>3) for 267 patients (12%) among 2148 for whom this information was available. The mean or median age of the patients was below or equal to five years in 24 studies and above five years for one study; this data was not available in eight articles.

### Detection of Vesicoureteral Reflux of any Grade

The characteristics of the studies concerning the prediction of reflux of any grade are presented in Table 2. The overall indices according to the ultrasound criteria are summarized in table 3.

Pyelocaliceal dilation, ureter dilation, urinary tract dilation and any type of dilation had a statistically significant diagnostic odds ratio: 3.3 (CI 95%=1.5-7.2); 1.8 (CI 95%=1.0-3.0); 2.3 (CI 95%=1.6-3.5) and 2.3 (CI 95%=1.6-3.5) respectively.

Only the “ureter dilation” ultrasound criterion showed low heterogeneity (Table 4). No factor explaining the heterogeneity concerning the pyelocaliceal dilation was found. However, concerning the urinary tract dilation or any type of dilation, the heterogeneity was significant and explained by a statistically significant threshold effect (Table 4). The studies differed significantly according to the thresholds chosen by the authors of the studies in order to define dilation. These thresholds were rarely clearly explained in the articles, but the Littenberg and Moses regression model used made it possible to make an overall estimation (ref. 31, 32). The so-called “univariate” regression models did not show any other source of heterogeneity (Table 4).

The “renal size” ultrasound criterion had a diagnostic odds ratio equal to 4.0 (CI 95%=1.6-9.6), with 76% heterogeneity, explained by a statistically significant threshold effect (Spearman coefficient=0.4; p<0.01; Table 4).

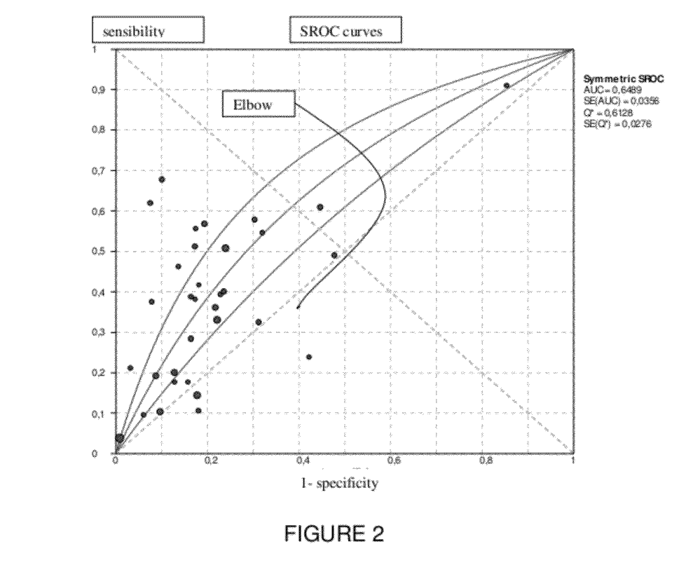

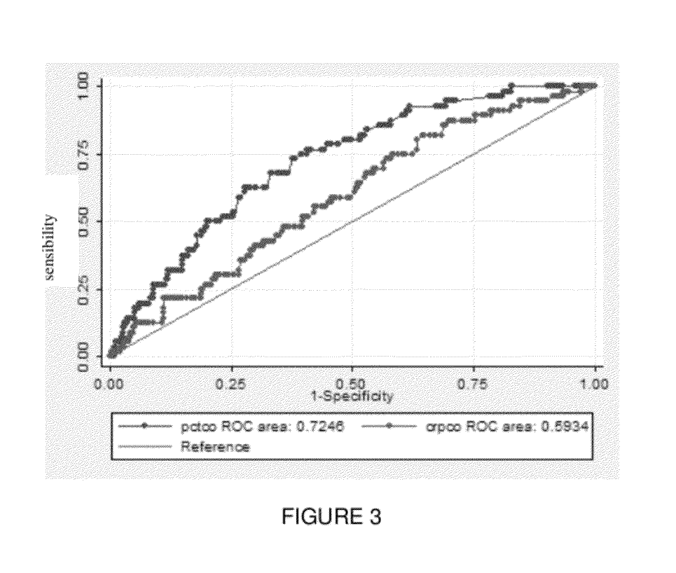

The analysis of the studies that did not include specific ultrasound criteria showed a significant diagnostic odds ratio (2.0; CI 95%=1.5-2.7) for the “unspecified abnormal ultrasound” variable, with significant heterogeneity (p=0.03, 12=44%; FIG. 3 and Table 4), explained by a threshold effect (Spearman coefficient=0.7; p<0.01; Table 4) and by the differences in populations included in the studies (diagnostic odds ratio adjusted for populations=1.5; CI 95%=1.0-2.5; p<0.05). The threshold effect was confirmed by the observation of the SROC “elbow” curve (FIG. 2).

The overall analysis of all of the studies, all ultrasound criteria combined, found a statistically significant diagnostic odds ratio (2.5; CI 95%=1.8-3.3), with 68% heterogeneity (Table 4), due, inter alia, to the different ultrasound criteria used (diagnostic odds ratio adjusted for ultrasound criteria=1.2; CI 95%=1.1-1.3; p<0.05) according to the so-called “univariate” meta-regression model.

Two ultrasound criteria (thickening of the pyelic wall and thickening of the pyelic and/or ureter wall) could not be studied in the meta-analysis because they concerned only one study each (Ref. 47, 54) (Table 2).

### Prediction of High-Grade Reflux

Ten studies included, representing a total of 1857 patients, had data available concerning the standard renal ultrasound and high-grade vesicoureteral reflux (Ref. 35, 26, 28, 37, 39, 46, 50, 51, 53 and 60). The characteristics of these studies are presented in Table 5. The overall indices according to the ultrasound criteria are summarized in Table 6.

All of the criteria (including any dilation, and the renal ultrasound, all criteria combined) showed a statistically significant diagnostic odds ratio (Table 6). However, only the results concerning ureter dilation and renal size did not have any statistically significant heterogeneity (Table 7). The heterogeneity for the pyelocaliceal dilation was explained by a threshold effect (Spearman coefficient=−1.0; p<0.01; Table 7). The results for the other ultrasound criteria did not appear to be subject to a threshold effect.

The so-called “univariate” regression model showed the consecutive or non-consecutive recruitment as a possible source of heterogeneity for the overall analysis (adjusted diagnostic odds ratio=84; CI 95%=1.7-4.1; Table 7).

Three ultrasound criteria (pyelic dilation, thickening of the pyelic wall and thickening of the pyelic and/or ureter wall) could not be studied in the meta-analysis because they concerned either no study (thickening of the pyelic and/or ureter wall) or only one study (26) (pyelic dilation, results in Table 4).

### Publication Bias

Visually, the funnel plot showed a symmetrical distribution of the studies and did not suggest that studies were missing for vesicoureteral reflux of any grade and of high grade in particular in the left-hand portion of the study. The Egger regression test did not show any publication bias (p>0.1) for the vesicoureteral reflux of any grade and of high grade.

### Main Results

The question of the prediction of vesicoureteral reflux by renal ultrasound appeared to be a question of benefit since 103 studies (of which 33 were included in the end) were published on the subject between 1985 and 2006 and the overall analysis showed a statistically significant diagnostic odds ratio for the prediction of vesicoureteral reflux of any grade and of high grade. However, during this overall analysis of the benefit of renal ultrasound in the prediction of vesicoureteral reflux (of any grade and of high grade), regardless of the ultrasound criterion, the high degree of heterogeneity suspected in the non-systematic preliminary review was confirmed (Ref. 62) and associated in particular with the multiple possible ultrasound criteria.

This example shows that the best ultrasound criterion for the prediction of vesicoureteral reflux of any grade and of high grade appears to be ureter dilation.

Indeed, the diagnostic odds ratio was 1.8 (CI 95%=1.0-3.0), with low heterogeneity and without a threshold effect for the prediction of vesicoureteral reflux of any grade. The diagnostic odds ratio reached 5.5 (CI 95%=1.3-22) with a heterogeneity at the limit of significance for the prediction of high-grade vesicoureteral reflux. This result confirms the “natural” impression of the clinician that this criterion is relatively unaffected by a threshold effect: the visibility on the ultrasound of a ureter in a child is considered by many to be pathological, regardless of its degree of dilation (Ref. 63).

The overall sensitivity of this ultrasound criterion for the prediction of vesicoureteral reflux of any grade and of high grade was poor: 13% (CI 95%=10-157) and 17% (CI 95%=9-29) respectively. Its specificity was, however beneficial both for the prediction of vesicoureteral reflux of any grade and of high grade: 92% (CI 95%=90-93) and 96% (CI 95%=94-98) respectively.

The results concerning the other ultrasound criteria cannot be used due to their high degree of heterogeneity, whether for the prediction of vesicoureteral reflux of any grade or that of high grade.

As shown by the large number of studies included in this meta-analysis, many teams were interested in renal ultrasound as a predictor of vesicoureteral reflux, which probably reflects the belief by clinicians in the benefit of this examination.

However, it is difficult to define with certainty, with the data currently available, whether the renal ultrasound is a good tool for predicting vesicoureteral reflux and which is the best ultrasound criterion for that.

As shown in this example, ureter dilation is the only ultrasound criterion significantly associated with vesicoureteral reflux of any grade and of high grade with low heterogeneity, and therefore appears to be the best ultrasound criterion for predicting vesicoureteral reflux of any grade and of high grade in the child after a first urinary tract infection.

TABLE 1
methodological quality of the studies included

| | | Centres |
|---|---|---|
| | | completing |
| | | this |
| Methodological item | Information sought in each study | criterion (n) |
| Did the test result influence | Did all of the patients undergo cystography regardless | 18 |
| whether or not the reference | of the ultrasound result? | |
| examination was performed (“work- | | |
| up” bias)? | | |
| Was the reference examination | Was the diagnosis of vesicoureteral reflux considered | 7 |
| interpreted blindly from the test | independently of the ultrasound result? | |
| result? | | |
| Description of the reference | Were sufficient details regarding the mode of diagnosis | 33 |
| examination | of vesicoureteral reflux available? | |
| | Was the reference test objective, unbiased, appropriate | 28 |
| | and available for all patients or for a large | |
| | percentage? | |
| Description of the test | Were sufficient details on the ultrasound test | 10 |
| | available? | |
| | Was the measurement of ultrasound criteria available | 31 |
| | for all of the patients or for a large percentage? | |
| Description of the study | Were there sufficient details concerning the | 29 |
| population | characteristics of the patients included? | |
| Patient recruitment method | Were the patients included prospectively and | 5 |
| | consecutively? | |
| | Was the sample representative and were the patients at | 29 |
| | a common stage in their disease (urinary tract | |
| | infection)? | |

TABLE 2
Characteristics and indices of studies for prediction of vesicoureteral reflux of any grade

| | | | | | | | | | | | |
|---|---|---|---|---|---|---|---|---|---|---|---|
| | | | Patients | Study | | Prevalence | Sensitivity | Specificity | PLR | NLR | DOR |
| Study | Year | Country | (n) | type | Population | of VUR | (CI 95%) | (CI 95%) | (CI 95%) | (CI 95%) | (CI 95%) |

Group 1: pyelic dilation

| | | | | | | | | | | | |
|---|---|---|---|---|---|---|---|---|---|---|---|
| Davey | 1997 | US | 445 | R | 2 | 38 | 14 | 82 | 0.8 | 1.0 | 0.8 |
| | | | | | | | (10-21) | (77-87) | (0.5-1.3) | (1.0-1.1) | (0.5-1.3) |
| Evans | 1999 | US | 57 | P | 2 | 17 | 11 | 82 | 0.6 | 1.1 | 0.5 |
| | | | | | | | (1-33) | (73-89) | (0.1-2.3) | (0.9-1.3) | (0.1-2.5) |
| Tsai | 2004 | Taiwan | 114 | R | 0 | 29 | 19 | 91 | 2.2 | 0.9 | 2.4 |
| | | | | | | | (9-33) | (86-95) | (1.0-4.6) | (0.8-1.0) | (1.0-5.9) |

Group 2: pyelocaliceal dilation

| | | | | | | | | | | | |
|---|---|---|---|---|---|---|---|---|---|---|---|
| Alon | 1986 | Israel | 81 | P | 1 | 22 | 22 | 89 | 2.0 | 0.9 | 2.3 |
| | | | | | | | (6-48) | (78-95) | (0.7-6.1) | (0.7-1.1) | (0.6-8.9) |
| Baronciani | 1985 | Italy | 74 | R | 1 | 28 | 62 | 93 | 8.2 | 0.4 | 20 |
| | | | | | | | (38-82) | (82-98) | (3.0-22) | (0.2-0.7) | (5.2-77) |
| Gelfand | 2000 | US | 743 | R | 1 | 25 | 2 | 99 | 3.0 | 1.0 | 3.0 |
| | | | | | | | (1-5) | (98-100) | (0.8-12) | (1.0-1.0) | (0.8-12) |
| Goldman | 2000 | Israel | 44 | P | 0 | 48 | 38 | 83 | 2.2 | 0.7 | 2.9 |
| | | | | | | | (18-62) | (61-95) | (0.8-6.2) | (0.5-1.1) | (0.7-12) |
| Leroy | 2005 | France | 149 | R | 0 | 25 | 22 | 80 | 1.1 | 1.0 | 1.1 |
| | | | | | | | (10-38) | (72-87) | (0.5-2.3) | (0.8-1.2) | (0.5-2.8) |
| Leroy | 2007 | Europe | 384 | P | 0 | 25 | 7 | 98 | 3.6 | 0.9 | 3.8 |
| | | | | | | | (3-15) | (96-99) | (1.4-4.9) | (0.8-1.0) | (1.5-11) |

Group 3: ureter dilation

| | | | | | | | | | | | |
|---|---|---|---|---|---|---|---|---|---|---|---|
| Alon | 1986 | Israel | 81 | P | 1 | 22 | 22 | 94 | 3.5 | 0.8 | 4.2 |
| | | | | | | | (6-48) | (85-98) | (1.0-13) | (0.6-1.1) | (0.9-19) |
| Gelfand | 2000 | US | 743 | R | 1 | 25 | 2 | 100 | 9.0 | 1.0 | 9.1 |
| | | | | | | | (1-5) | (100-100) | (0.9-86) | (1.0-1.0) | (0.9-88) |
| Kenney | 2002 | England | 144 | R | 1 | 23 | 4 | 78 | 1.7 | 0.8 | 2.0 |
| | | | | | | | (2-5) | (71-85) | (1.1-2.6) | (0.7-1.0) | (1.1-3.9) |
| Leroy | 2005 | France | 149 | R | 0 | 25 | 1 | 92 | 1.0 | 1.0 | 1.0 |
| | | | | | | | (1-2) | (85-96) | (0.3-3.5) | (0.9-1.1) | (0.3-3.9) |
| Leroy | 2007 | Europe | 384 | P | 0 | 25 | 20 | 83 | 1.2 | 1.0 | 1.2 |
| | | | | | | | (13-30) | (78-87) | (0.7-7.9) | (0.9-1.1) | (0.7-2.2) |

Group 4: urinary tract dilation

| | | | | | | | | | | | |
|---|---|---|---|---|---|---|---|---|---|---|---|
| ALON | 1986 | Israel | 81 | P | 1 | 22 | 3 | 89 | 3.0 | 0.8 | 4.0 |
| | | | | | | | (1-6) | (78-95) | (1.2-7.8) | (0.6-1.1) | (1.1-14) |
| ALON | 1999 | Israel | 100 | R | 0 | 38 | 2 | 97 | 6.5 | 0.8 | 8.0 |
| | | | | | | | (1-4) | (89-100) | (1.5-29) | (0.7-1.0) | (1.6-4.0) |
| CLEPER | 2004 | Israel | 64 | R | 0 | 20 | 5 | 86 | 3.4 | 0.6 | 5.4 |
| | | | | | | | (2-7) | (74-94) | (1.4-8.3) | (0.4-1.0) | (1.4-21) |
| FORESMAN | 2001 | US | 139 | R | 1 | 35 | 4 | 66 | 1.2 | 0.9 | 1.3 |
| | | | | | | | (3-6) | (55-75) | (0.8-1.8) | (0.7-1.2) | (0.6-2.7) |
| GELFAND | 2000 | US | 743 | R | 1 | 25 | 0 | 99 | 4.2 | 1.0 | 4.3 |
| | | | | | | | (0-1) | (98-100) | (1.3-13) | (0.9-1.0) | (1.4-14) |
| HOBERMAN | 2003 | US | 302 | P | 0 | 39 | 10 | 90 | 1.0 | 1.0 | 1.1 |
| | | | | | | | (1-17) | (85-94) | (0.5-2.1) | (0.9-1.0) | (0.5-2.3) |
| LEROY | 2005 | France | 149 | R | 0 | 25 | 24 | 78 | 1.1 | 1.0 | 1.1 |
| | | | | | | | (12-41) | (69-85) | (0.6-2.1) | (0.8-1.2) | (0.5-2.7) |
| LEROY | 2007 | Europe | 393 | P | 0 | 25 | 25 | 81 | 1.3 | 0.9 | 1.4 |
| | | | | | | | (17-35) | (76-85) | (0.9-2.0) | (0.8-1.1) | (0.8-2.4) |
| MAHANT | 2002 | Canada | 162 | R | 0 | 22 | 40 | 76 | 1.7 | 0.8 | 2.1 |
| | | | | | | | (24-58) | (68-84) | (1.0-2.8) | (0.6-1.0) | (1.0-4.8) |
| MUENSTER* | 2002 | US | 193 | R | 2 | 18 | 51 | 76 | 2.1 | 0.6 | 3.3 |
| | | | | | | | (38-63) | (71-81) | (1.6-2.9) | (0.5-0.8) | (1.9-5.6) |
| OOSTENBRINK | 2000 | Netherlands | 140 | R | 0 | 26 | 57 | 81 | 2.9 | 0.5 | 5.4 |
| | | | | | | | (40-73) | (72-88) | (1.8-4.7) | (0.4-0.8) | (2.4-12) |
| SMELLIE | 1995 | England | 58 | R | 1 | 62 | 19 | 73 | 0.7 | 1.1 | 0.6 |
| | | | | | | | (8-36) | (50-89) | (0.3-1.8) | (0.8-1.5) | (0.2-2.2) |
| TASKINEN | 2005 | Finland | 61 | R | 0 | 18 | 55 | 68 | 1.7 | 0.7 | 2.5 |
| | | | | | | | (23-83) | (54-80) | (0.9-3.3) | (0.3-1.3) | (0.7-9.5) |
| ZOCCHI | 1988 | Italy | 117 | R | 1 | 11 | 19 | 100 | 42 | 0.8 | 53 |
| | | | | | | | (4-46) | (96-100) | (2.3-777) | (0.6-1.0) | (2.6-1075) |

Group 5: thickening of the pyelic wall

| | | | | | | | | | | | |
|---|---|---|---|---|---|---|---|---|---|---|---|
| MORIN | 1999 | France | 70 | R | 0 | 31 | 55 | 38 | 0.9 | 1.2 | 0.7 |
| | | | | | | | (34-75) | (24-51) | (0.6-1.4) | (0.7-2.2) | (0.3-2.0) |

Group 6: thickening of the pyelic and/or ureter wall

| | | | | | | | | | | | |
|---|---|---|---|---|---|---|---|---|---|---|---|
| GELFAND | 2000 | US | 743 | R | 1 | 25 | 4 | 96 | 1.1 | 1.0 | 1.1 |
| | | | | | | | (1-7) | (95-98) | (0.5-2.5) | (1.0-1.0) | (0.5-2.6) |

Group 7: RENAL SIZE

| | | | | | | | | | | | |
|---|---|---|---|---|---|---|---|---|---|---|---|
| ALON | 1986 | Israel | 81 | P | 1 | 22 | 11 | 98 | 7.0 | 0.9 | 7.8 |
| | | | | | | | (1-3) | (92-100) | (0.7-73) | (0.8-1.1) | (0.7-91) |
| LEROY | 2005 | France | 149 | R | 0 | 25 | 8 | 89 | 0.8 | 1.0 | 0.7 |
| | | | | | | | (2-22) | (82-94) | (0.2-2.5) | (0.9-1.2) | (0.2-2.8) |
| LEROY | 2007 | France | 384 | P | 0 | 31 | 82 | 50 | 1.6 | 0.4 | 4.5 |
| | | | | | | | (60-95) | (35-65) | (1.2-2.3) | (0.1-0.9) | (1.3-15) |
| MUENSTER* | 2002 | US | 193 | R | 2 | 18 | 29 | 91 | 3.3 | 0.8 | 4.2 |
| | | | | | | | (19-41) | (88-94) | (2.0-5.5) | (0.7-0.9) | (2.2-8.1) |
| PERATONER* | 2005 | Italy | 77 | R | 1 | 67 | 68 | 90 | 6.7 | 0.4 | 19 |
| | | | | | | | (55-79) | (82-95) | (3.5-13) | (0.3-0.5) | (7.9-44) |

Group 8: unspecified abnormal renal ultrasound

| | | | | | | | | | | | |
|---|---|---|---|---|---|---|---|---|---|---|---|
| ALMEIDA | 1994 | Portugal | 147 | R | 2 | 30 | 51 | 83 | 3.0 | 0.6 | 5.0 |
| | | | | | | | (36-67) | (74-89) | (1.8-4.9) | (0.4-0.8) | (2.3-11) |
| ALON | 1986 | Israel | 81 | P | 1 | 22 | 56 | 83 | 3.2 | 0.5 | 5.9 |
| | | | | | | | (31-79) | (71-91) | (1.6-6.3) | (0.3-0.9) | (1.9-18) |
| BOUDAILLEZ | 1989 | France | 92 | R | 0 | 23 | 24 | 58 | 0.6 | 1.3 | 0.4 |
| | | | | | | | (8-47) | (45-69) | (0.3-1.3) | (1.0-1.8) | (0.1-1.3) |
| CALISTI | 2005 | Italy | 147 | P | 0 | 31 | 58 | 70 | 1.9 | 0.6 | 3.1 |
| | | | | | | | (42-72) | (60-78) | (1.3-2.8) | (0.4-0.9) | (1.5-6.5) |
| DAVID | 1998 | France | 152 | R | 0 | 44 | 28 | 84 | 1.7 | 0.9 | 2.0 |
| | | | | | | | (18-41) | (74-91) | (6.9-3.2) | (0.7-1.0) | (0.9-4.4) |
| DIPIETRO | 1997 | US | 70 | R | 1 | 30 | 10 | 94 | 1.6 | 1.0 | 1.6 |
| | | | | | | | (1-30) | (83-99) | (0.3-8.6) | (0.8-1.1) | (0.2-10) |
| DURATRAVE* | 1997 | Spain | 24 | R | 0 | 54 | 18 | 87 | 1.4 | 1.0 | 1.4 |
| | | | | | | | (4-43) | (70-96) | (0.3-5.4) | (0.7-1.2) | (0.3-7.4) |
| FORESMA | 2001 | US | 139 | R | 1 | 35 | 49 | 52 | 1.0 | 1.0 | 1.0 |
| | | | | | | | (34-64) | (41-63) | (0.7-1.5) | (0.7-1.4) | (0.5-2.1) |
| HONKINEN | 1986 | Finland | 76 | R | 1 | 37 | 39 | 77 | 1.7 | 0.8 | 2.2 |
| | | | | | | | (22-59) | (63-88) | (0.0-3.4) | (0.6-1.1) | (0.8-6.0) |
| LEROY | 2005 | France | 149 | R | 0 | 25 | 32 | 69 | 1.0 | 1.0 | 1.1 |
| | | | | | | | (18-50) | (59-77) | (0.6-1.8) | (0.8-1.3) | (0.5-2.3) |
| LEROY | 2007 | Europe | 393 | P | 0 | 25 | 33 | 78 | 1.5 | 0.9 | 1.7 |
| | | | | | | | (24-43) | (73-82) | (1.0-2.1) | (0.7-1.0) | (1.0-2.8) |
| MAGE | 1989 | France | 122 | R | 0 | 25 | 39 | 84 | 2.1 | 0.7 | 3.2 |
| | | | | | | | (22-58) | (74-91) | (1.2-4.5) | (0.5-1.0) | (1.3-8.0) |
| MORIN | 1999 | France | 70 | R | 0 | 31 | 91 | 15 | 1.1 | 0.6 | 1.7 |
| | | | | | | | (71-99) | (6-28) | (0.9-1.3) | (0.1-2.8) | (0.3-9.0) |
| SMELLLIE | 1995 | England | 58 | R | 1 | 62 | 42 | 82 | 2.3 | 0.7 | 3.2 |
| | | | | | | | (26-59) | (60-95) | (0.9-6.0) | (0.5-1.0) | (0.9-11) |
| TAN | 1988 | Singapore | 55 | R | 1 | 31 | 18 | 54 | 1.2 | 1.0 | 1.1 |
| | | | | | | | (4-43) | (69-94) | (0.3-4.0) | (0.8-1.3) | (0.3-5.2) |
| YEN | 1999 | Taiwan | 187 | R | 0 | 25 | 61 | 55 | 1.4 | 0.7 | 1.9 |
| | | | | | | | (45-75) | (47-64) | (1.0-1.8) | (0.5-1.0) | (1.0-3.8) |
| ZAMIR | 2004 | Israel | 255 | P | 0 | 18 | 20 | 87 | 1.6 | 0.9 | 1.7 |
| | | | | | | | (10-35) | (82-91) | (0.8-3.1) | (0.8-1.1) | (0.7-3.9) |
| ZOCCHI | 1988 | Italy | 117 | R | 1 | 11 | 38 | 92 | 4.7 | 0.7 | 7.0 |
| | | | | | | | (15-65) | (85-97) | (1.9-12) | (0.5-1.0) | (2.0-24) |

Abbreviations: DOR, diagnostic odds ratio; CI, confidence interval, P, prospective, R, retrospective; NLR, negative likelihood ratio; PLR, positive likelihood ratio; VUR, vesicoureteral reflux. Population: 0, first urinary tract infection; 1, urinary tract infection (without specification of the number of prior urinary tract infections); 2, patients having undergone a renal ultrasound and cystography within a period of less than six months.

TABLE 3
Overall indices for each ultrasound criterion for the prediction of vesicoureteral reflux of any grade

| | | | n* | n* | | | | | | | |
|---|---|---|---|---|---|---|---|---|---|---|---|
| Ultrasound | n | | test+ | test− | Sensitivity | Specificity | PLR | NLR | DOR | P | |
| criterion | studies | n* | and VUR | and VUR | (CI 95%) | (CI 95%) | (CI 95%) | (CI 95%) | (CI 95%) | (Q test) | I² |
| Pyelic | 3 | 796 | 36 | 83 | 15 | 85 | 1.1 | 1.0 | 1.1 | 0.6 | 63 |
| dilation | | | | | (11-20) | (82-88) | (0.5-2.3) | (0.9-1.1) | (0.5-2.6) | | |
| Pyelocaliceal | 6 | 1475 | 44 | 47 | 12 | 96 | 2.6 | 0.9 | 3.3 | 0.03 | 59 |
| dilation | | | | | (9-15) | (94-97) | (1.4-4.9) | (0.8-1.0) | (1.5-7.2) | | |
| Ureter | 5 | 1565 | 51 | 96 | 13 | 92 | 1.6 | 0.9 | 1.8 | 0.2 | 30 |
| dilation | | | | | (10-17) | (90-93) | (1.0-2.4) | (0.9-1.0) | (1.0-3.0) | | |
| Urinary tract | 14 | 2898 | 179 | 300 | 24 | 86 | 1.8 | 0.9 | 2.3 | 0.003 | 59 |
| dilation | | | | | (21-27) | (84-87) | (1.4-2.4) | (0.8-0.9) | (1.6-3.5) | | |
| Any urinary | 20 | 4020 | 258 | 423 | 23 | 86 | 1.8 | 0.9 | 2.3 | <0.001 | 68 |
| tract | | | | | (21-26) | (84-87) | (1.4-2.3) | (0.8-0.9) | (1.6-3.3) | | |
| dilation** | | | | | | | | | | | |
| Thickening of | 1 | 70 | 12 | 30 | 55 | 38 | 0.9 | 1.2 | 0.7 | | |
| the pyelic | | | | | (34-75) | (24-51) | (0.6-1.4) | (0.7-2.2) | (0.3-2.0) | | |
| wall | | | | | | | | | | | |
| Thickening of | 1 | 743 | 8 | 21 | 4 | 96 | 1.1 | 1.0 | 1.1 | | |
| the pyelic | | | | | (1-7) | (95-98) | (0.5-2.5) | (1.0-1.0) | (0.5-2.6) | | |
| and/or ureter | | | | | | | | | | | |
| wall | | | | | | | | | | | |
| Renal size | 6 | 1224 | 96 | 88 | 31 | 90 | 2.5 | 0.8 | 4.0 | 0.001 | 76 |
| | | | | | (26-37) | (88-92) | (1.4-4.7) | (0.6-1.0) | (1.6-9.6) | | |
| Abnormal† | 18 | 1958 | 260 | 29 | 40 | 75 | 1.6 | 0.8 | 2.0 | 0.03 | 44 |
| renal | | | | | (36-43) | (73-77) | (1.3-2.0) | (0.8-0.9) | (1.5-2.7) | | |
| ultrasound | | | | | | | | | | | |
| Any | 33 | 5595 | 492 | 736 | 31 | 82 | 1.8 | 0.8 | 2.5 | <0.001 | 68 |
| abnormality | | | | | (29-34) | (81-83) | (1.5-2.2) | (0.7-0.9) | (1.8-3.3) | | |

*Number of patients or renal units (according to the studies).
**For this analysis, we took into consideration the 14 studies having studied dilation of the urinary tract, and the six studies not included in this sub-group but having studied pyelic or caliceal or ureter dilation.
†So-called abnormal ultrasound, unspecified.
Abbreviations: DOR, diagnostic odds ratio; CI, confidence interval; NLR, negative likelihood ratio; PLR, positive likelihood ratio.

TABLE 4
Studies of factors capable of explaining heterogeneity in the prediction of vesicoureteral reflux of any grade by renal ultrasound

| | | | | | | | Meta-regression‡ | | | | | |
|---|---|---|---|---|---|---|---|---|---|---|---|---|
| | | DOR | Heterogeneity | | Threshold effect | | | | | Missing | | Ultrasound |
| Criteria | n* | (CI 95%) | p** | I² | Spearman† | p | R/P | Blind | Consecutivity | data | Population | criteria |
| Pyelic | 3 | 1.1 | 0.6 | 63 | −1.0 | <0.01 | — | — | — | — | — | — |
| dilation | | (0.5-2.6) | | | | | | | | | | |
| Pyelocaliceal | 6 | 3.3 | 0.03 | 59 | 0.5 | 0.3 | — | — | — | — | — | — |
| dilation | | (1.5-7.2) | | | | | | | | | | |
| Ureter | 5 | 1.8 | 0.2 | 30 | 0.7 | 0.2 | — | — | — | — | — | — |
| dilation | | (1.0-3.0) | | | | | | | | | | |
| Urinary tract | 14 | 2.3 | <0.01 | 59 | 0.6 | 0.03 | 1.7 | 1.6 | 0.8 | 0.5 | 1.1 | — |
| dilation | | (1.6-3.5) | | | | | (0.7-4.2) | (0.6-4.2) | (0.2-3.1) | (0.1-2.3) | (0.6-2.2) | |
| Any dilation | 20 | 2.3 | <0.01 | 68 | 0.4 | 0.07 | 1.7 | 1.8 | 1.2 | 1.8 | 0.8 | 1.2 |
| | | (1.6-3.3) | | | | | (0.7-4.2) | (0.8-4.1) | (0.4-3.6) | (0.5-6.7) | (0.5-1.4) | (0.9-1.5) |
| Renal size | 6 | 4.0 | <0.01 | 76 | 0.4 | <0.01 | — | — | — | — | — | — |
| | | (1.6-9.6) | | | | | | | | | | |
| Abnormal | 18 | 2.0 | 0.03 | 44 | 0.7 | <0.01 | 0.8 | 0.9 | 1.5 | — | 1.5 | — |
| ultrasound | | (1.5-2.7) | | | | | (0.4-1.6) | (0.4-1.9) | (0.8-2.7) | | (1.0-2.5) | |
| Any | 33 | 2.5 | <0.01 | 68 | 0.5 | <0.01 | 1.4 | 1.4 | 1.4 | — | 1.1 | 1.2 |
| abnormality | | (1.8-3.3) | | | | | (0.7-2.8) | (0.7-2.9) | (0.8-2.6) | | (0.7-1.7) | (1.1-1.3) |

*Number of studies,
**Q test significance coefficient,
†Spearman correlation coefficient,
‡DOR adjusted in the so-called “univariate” meta-regression model (when n ≧ 10 studies), expressed as follows: DOR (CI 95%).
Abbreviations: DOR, diagnostic odds ratio; CI, confidence interval; R, retrospective study; P, prospective study.

TABLE 5
Characteristics and indices of studies included for the prediction of high-grade vesicoureteral reflux

| | | | | | | | | | | | |
|---|---|---|---|---|---|---|---|---|---|---|---|
| | | | Patients | Study | | Prevalence | Sensitivity | Specificity | PLR | NLR | DOR |
| Study | Year | Country | (n) | type | Population | of VUR | (CI 95%) | (CI 95%) | (CI 95%) | (CI 95%) | (CI 95%) |

Group 1: pyelic dilation

| | | | | | | | | | | | |
|---|---|---|---|---|---|---|---|---|---|---|---|
| Tsai | 2004 | Taiwan | 114 | R | 0 | 12 | 25 | 91 | 2.9 | 0.8 | 3.5 |
| | | | | | | | (10-40) | (87-95) | (1.4-6.1) | (0.7-1.0) | (1.4-9.0) |

Group 2: pyelocaliceal dilation

| | | | | | | | | | | | |
|---|---|---|---|---|---|---|---|---|---|---|---|
| Alon | 1986 | Israel | 81 | P | 1 | 11 | 44 | 90 | 4.6 | 0.6 | 7.4 |
| | | | | | | | (14-79) | (81-96) | (1.7-12) | (0.3-1.1) | (1.6-34) |
| Baronciani | 1985 | Italy | 74 | R | 1 | 16 | 100 | 92 | 11 | 0.0 | 261 |
| | | | | | | | (74-100) | (82-97) | (4.9-25) | (0.0-0.6) | (14-5039) |
| Leroy | 2005 | France | 149 | R | 0 | 9 | 29 | 81 | 1.5 | 0.9 | 1.7 |
| | | | | | | | (8-58) | (73-87 | (0.6-3.6) | (0.6-1.2) | (0.5-5.8) |
| Leroy | 2007 | Europe | 384 | P | 0 | 12 | 29 | 84 | 2.1 | 0.8 | 2.7 |
| | | | | | | | (8-58) | (79-88) | (1.3-3.4) | (0.5-1.0) | (1.3-5.4) |

Group 3: ureter dilation

| | | | | | | | | | | | |
|---|---|---|---|---|---|---|---|---|---|---|---|
| Alon | 1986 | Israel | 81 | P | 1 | 11 | 44 | 94 | 8.0- | 0.6 | 14 |
| | | | | | | | (14-79) | (86-99) | 2.4-27) | (0.3-1.1) | (2.6-71) |
| Leroy | 2005 | France | 149 | R | 0 | 9 | 7 | 92 | 0.9 | 1.0 | 0.9 |
| | | | | | | | (0-34) | (86-96) | (0.1-6.3) | (0.9-1.2) | (0.1-7.3) |
| Leroy | 2007 | Europe | 384 | P | 0 | 12 | 15 | 98 | 7.1 | 0.9 | 8.1 |
| | | | | | | | (6-29) | (96-99) | (2.5-20) | (0.8-1.0) | (2.6-26) |

Group 4: urinary tract dilation

| | | | | | | | | | | | |
|---|---|---|---|---|---|---|---|---|---|---|---|
| Alon | 1986 | Israel | 81 | P | 1 | 11 | 67 | 90 | 6.9 | 0.4 | 19 |
| | | | | | | | (30-93) | (81-96) | (3.0-16) | (0.1-0.9) | (3.8-91) |
| Foresman | 2001 | US | 139 | R | 1 | 17 | 44 | 65 | 1.2 | 0.9 | 1.4 |
| | | | | | | | (23-66) | (55-73) | (0.7-2.1) | (0.6-1.3) | (0.6-3.5) |
| Hoberman | 2003 | US | 302 | P | 0 | 17 | 20 | 90 | 2.1 | 0.9 | 2.3 |
| | | | | | | | (10-34) | (85-94) | (1.0-4.2) | (0.8-1.0) | (1.0-5.4) |
| Leroy | 2005 | France | 149 | R | 0 | 9 | 36 | 79 | 1.7 | 0.8 | 2.0 |
| | | | | | | | (13-65) | (71-85) | (0.8-3.6) | (0.5-1.2) | (0.6-6.5) |
| Leroy | 2007 | Europe | 388 | P | 0 | 12 | 40 | 82 | 2.2 | 0.7 | 3.0 |
| | | | | | | | (26-56) | (77-86) | (1.5-3.4) | (0.6-0.9) | (1.6-5.8) |
| Mahant | 2002 | Canada | 162 | R | 0 | 8 | 45 | 75 | 1.8 | 0.7 | 2.5 |
| | | | | | | | (19-75) | (67-81) | (0.9-3.3) | (0.4-1-2) | (0.8-7.9) |
| Muensterer* | 2002 | US | 193 | R | 2 | 6 | 78 | 74 | 3.1 | 0.3 | 10 |
| | | | | | | | (56-93) | (70-79) | (2.3-4.0) | (0.1-0.6) | (3.8-29) |

Group 5: renal size

| | | | | | | | | | | | |
|---|---|---|---|---|---|---|---|---|---|---|---|
| Alon | 1986 | Israel | 81 | P | 1 | 11 | 11 | 97 | 4.0 | 0.9 | 4.4 |
| | | | | | | | (3-48) | (90-100) | (0.4-39) | (0.7-1.2) | (0.4-9154) |
| Leroy | 2005 | France | 149 | R | 0 | 9 | 21 | 91 | 2.4 | 0.9 | 2.8 |
| | | | | | | | (5-51) | (85-95) | (0.8-7.5) | (0.7-1.1) | (0.7-11) |
| Leroy | 2007 | Europe | 384 | P | 0 | 12 | 10 | 94 | 1.7 | 1.0 | 1.8 |
| | | | | | | | (3-23) | (91-97) | (0.6-4.9) | (0.9-1.1) | (0.6-5.6) |
| Muensterer* | 2002 | US | 193 | R | 2 | 6 | 48 | 90 | 4.7 | 0.6 | 8.0 |
| | | | | | | | (27-59) | (86-93) | (2.8-7.9) | (0.4-0.9) | (3.3-20) |

Group 6: unspecified abnormal renal ultrasound

| | | | | | | | | | | | |
|---|---|---|---|---|---|---|---|---|---|---|---|
| Alon | 1986 | Israel | 81 | P | 1 | 11 | 89 | 82 | 4.9 | 0.1 | 36 |
| | | | | | | | (52-100) | (71-90) | (2.9-8.5) | (0.0-0.9) | (4.2-316) |
| Foresman | 2001 | US | 139 | R | 1 | 17 | 48 | 52 | 1.0 | 1.0 | 1.0 |
| | | | | | | | (27-69) | (42-61) | (0.6-1.6) | (0.7-1.5) | (0.4-2.4) |
| Leroy | 2005 | France | 149 | R | 0 | 9 | 50 | 70 | 1.7 | 0.7 | 2.4 |
| | | | | | | | (23-77) | (62-78) | (0.9-3.0) | (0.4-1.2) | (0.8-7.2) |
| Leroy | 2007 | Europe | 388 | P | 0 | 12 | 44 | 78 | 2.0 | 0.7 | 2.8 |
| | | | | | | | (30-60) | (73-82) | (1.4-2.9) | (0.5-0.9) | (1.5-5.2) |
| Muensterer* | 2002 | US | 193 | R | 2 | 6 | 91 | 68 | 2.8 | 0.1 | 22 |
| | | | | | | | (72-99) | (62-72) | (2.3-3.4) | (0.0-0.5) | (5.0-95) |
| Zamir | 2004 | Israel | 255 | P | 0 | 6 | 21 | 86 | 1.6 | 0.9 | 1.7 |
| | | | | | | | (5-51) | (81-90) | (0.5-4.5) | (0.7-1.2) | (0.5-6.5) |

Abbreviations: DOR, diagnostic odds ratio; CI, confidence interval, P, prospective, R, retrospective; NLR, negative likelihood ratio; PLR, positive likelihood ratio; VUR, vesicoureteral reflux. Population: 0, first urinary tract infection; 1, urinary tract infection (without specification of the number of prior urinary tract infections); 2, patients having undergone a renal ultrasound and cystography within a period of less than six months.
*Studies in which the data was analyzed for each renal unit.

TABLE 6
Overall indices for each ultrasound criterion for the prediction of high-grade vesicoureteral reflux

| | | | n* | n* | | | | | | | |
|---|---|---|---|---|---|---|---|---|---|---|---|
| Ultrasound | n | | test+ | test− | Sensitivity | Specificity | PLR | NLR | DOR | P | |
| criterion | studies | n* | and VUR | and VUR | (CI 95%) | (CI 95%) | (CI 95%) | (CI 95%) | (CI 95%) | (Q test) | I² |
| Pyelic | 1 | 228 | 8 | 17 | 25 | 91 | 2.9 | 0.8 | 3.5 | | |
| dilation | | | | | (10-40) | (87-95) | (1.4-6.1) | (0.7-1.0) | (1.4-9.0) | | |
| Pyelocaliceal | 4 | 684 | 34 | 93 | 45 | 85 | 3.5 | 0.7 | 5.6 | 0.008 | 75 |
| dilation | | | | | (33-57) | (82-88) | (1.5-8.3) | (0.5-1.1) | (1.4-22) | | |
| Ureter | 3 | 610 | 11 | 22 | 17 | 96 | 4.7 | 0.9 | 5.5 | 0.1 | 57 |
| dilation | | | | | (9-29) | (94-98) | (1.5-15) | (0.7-1.1) | (1.3-22) | | |
| Urinary tract | 7 | 1540 | 73 | 288 | 41 | 79 | 2.3 | 0.7 | 3.3 | 0.03 | 58 |
| dilation | | | | | (34-49) | (77-81) | (1.6-3.2) | (0.6-0.9) | (1.9-6.0) | | |
| Any urinary | 9 | 1842 | 93 | 310 | 42 | 81 | 2.7 | 0.7 | 4.0 | 0.003 | 66 |
| tract | | | | | (36-49) | (79-83) | (1.8-3.9) | (0.6-0.9) | (2.2-7.3) | | |
| dilation** | | | | | | | | | | | |
| Renal size | 4 | 996 | 19 | 70 | 22 | 92 | 3.3 | 0.9 | 3.9 | 0.2 | 34 |
| | | | | | (14-32) | (90-94) | (1.9-5.7) | (0.7-1.1) | (1.7-8.6) | | |
| Abnormal† | 6 | 1398 | 70 | 337 | 55 | 74 | 2.1 | 0.7 | 3.6 | 0.002 | 74 |
| renal | | | | | (46-64) | (71-76) | (1.3-3.3) | (0.4-1.0) | (1.4-8.9) | | |
| ultrasound | | | | | | | | | | | |
| Any | 10 | 2097 | 106 | 415 | 45 | 78 | 2.4 | 0.7 | 3.8 | <0.001 | 70 |
| abnormality | | | | | (39-52) | (76-80) | (1.7-3.5) | (0.6-0.9) | (2.0-7.2) | | |

*Number of patients or renal units (according to the studies).
**For this analysis, we took into consideration the seven studies having studied urinary tract dilation, and the two studies not included in this sub-group but having studied either pyelic or pyelocaliceal dilation.
†So-called abnormal, unspecified ultrasound.
Abbreviations: DOR, diagnostic odds ratio; CI, confidence interval; NLR, negative likelihood ratio; PLR, positive likelihood ratio.

### Example 2

### Identification of the Serum Concentration of Procalcitonin as a Potential Marker for Vesicoureteral Reflux

The first experimental results concerning the identification of the serum concentration of procalcitonin as a potential marker for vesicoureteral reflux in a child from 1 month to 4 years of age having a first febrile urinary tract infection are described in the article “Procalcitonin as a predictor for vesico-ureteral reflux in children with urinary tract infection”. S. Leroy, C. Adamsbaum, E. Marc, F. Moulin, J. Raymond, D. Gendrel, G. Bréart, M. Chalumeau. Pediatrics 2005; 115: e706-709 (Ref. 52). For vesicoureteral reflux of any grade, a serum procalcitonin greater than or equal to 0.5 ng/ml has a sensitivity of 85% (CI 95%: 70-94), a specificity of 44% (CI 95%: 35-54), a positive predictive value of 34% (CI 95%: 22-48) and a negative predictive value of 90% (CI 95%: 74-97). For high-grade vesicoureteral reflux (greater than or equal to 3), a high serum procalcitonin (greater than or equal to 0.5 ng/ml) has a sensitivity of 92% (CI 95%: 65-99), a specificity of 44% (CI 95%: 35-54), a positive predictive value of 16% (CI 95%: 8-31) and a negative predictive value of 98% (CI 95%: 84-100).

These results were confirmed secondarily in a European multi-centre validation study (n=398 patients, 8 centres, 7 countries) presented in the article “Procalcitonin to reduce the number of unnecessary cystographies after a urinary tract infection in children: a European validation study. S. Leroy, C. Romanello, A. Galetto-Lacour, V. Smolkin, B. Korczowski, C. Rodrigo, D. Tuerlinckx, V. Gajdos, F. Moulin, M. Contardo, A. Gervaix, R. Halevy, B. Duhl, C. Prat, T. Vander Borght, L. Foix L′Hélias, F. Dubos, D. Gendrel, G. Bréart, M. Chalumeau. Journal of Pediatrics 2007; 150: 89-95. (Ref. 53). For vesicoureteral reflux of any grade, a serum procalcitonin greater than or equal to 0.5 ng/ml has a sensitivity of 75% (CI 95%: Ref. 68-85), a specificity of 43% (CI 95%: 37-48), a positive predictive value of 31% (CI 95%) and a negative predictive value of 84% (CI 95%). For high-grade vesicoureteral reflux (greater than or equal to 3), a high serum procalcitonin (greater than or equal to 0.5 ng/ml) has a sensitivity of 89% (CI 95%: 77-95), a specificity of 43% (CI 95%: 37-48), a positive predictive value of 19% (CI 95%: 14-25) and a negative predictive value of 96% (CI 95%: 91-99).

The specificity values and positive predictive values are not high. It therefore appears that the measurement of the serum concentration of procalcitonin is only a potential marker of vesicoureteral reflux, but does not make it possible to predict vesicoureteral reflux with a high degree of specificity.

### Example 3

### Example of Implementation of the First Embodiment of the Method for Predicting High-Grade Vesicoureteral Reflux after a First Febrile Urinary Tract Infection in Children

### A) Patients and Methods

### General Description

A secondary analysis of the data was performed with the single-centre cohort presented in Leroy et al., Pediatrics, “Procalcitonin as a predictor for vesico-ureteral reflux in children with urinary tract infection” (Ref. 14) and the prospective multi-centre data presented in Leroy et al. J. of Pediatrics, “Procalcitonin to reduce the number of unnecessary cystographies after a urinary tract infection in children: a European validation study” (Ref. 16) for the implementation of the method for predicting vesicoureteral reflux. More specifically, this example relates to high-grade vesicoureteral reflux (defined as a grade greater than or equal to 3) after a first febrile urinary tract infection in children. The criteria for inclusion and non-inclusion were as follows: all of the patients were between 1 month and 4 years of age with a first febrile urinary tract infection defined by a rectal temperature greater than or equal to 38° C. associated with bacteriuria. Patients with known uropathy or having received an antibiotic treatment within the preceding 48 hours were not included. Children with an isotopic cystography (which did not enable high-grade vesicoureteral reflux to be distinguished) were not included.

The vesicoureteral reflux was identified on a radiological cystography by a senior radiologist with a blind for potential predictors and graded from 0 to 5 according to the international classification (Ref. 21). As the objective was to predict high-grade vesicoureteral reflux, this predicted variable was therefore dichotomized as follows: reflux absent (grade 0) or low-grade (grades 1-2) by comparison with high-grade reflux, greater than or equal to 3).

### Predictive Variables

The serum procalcitonin of the patients was assayed prospectively by immunoluminometric analysis or by rapid semi-quantitative “PCT-Q” immunochromatographic assay (BRAHMS AG, Hennigsdorf, Germany) on admission of the patient.

The other predictive variables were ureter dilation, pyelocaliceal cavity dilation and renal size (only ultrasound criteria available in the database) present in the renal ultrasound performed at the time of diagnosis of the urinary tract infection by a senior paediatric radiologist, with a blind for the result of the procalcitonin assay.

### Co-Variables of Interest

The co-variables of interest were age, sex, CRP, family history of uropathy or first-degree febrile urinary tract infection. These co-variables were dichotomized by using thresholds already proposed in the prior art:
presence of family history of first-degree uropathy: 1, absence: 0 (Feather S. Vesicoureteric reflux: all in the genes? Lancet 1996; 348: 725-8. (Ref. 73); Chertin B., Puri P. Familial vesicoureteral reflux. J. Urol. 2003; 169: 1804-8. (Ref. 74));
age less than or equal to 1 year: 1, age greater than 1 year: 0 (Jacobson S. H., Hansson S., Jakobsson B. Vesico-ureteric reflux: occurrence and long-term risks. Acta Paediatr. 1999; 88: 22-30. (Ref. 75); Gelfand M. J., Koch B. L., Cordero G. G., Salmanzadeh A., Gartside P. S. Vesicoureteral reflux: subpopulations of patients defined by clinical variables. Pediatr. Radiol. 2000; 30: 121-4. (Ref. 76));
boy: 1, girl: 0 (Jakobsson B., Jacobson S. H., Hjalmas K. Vesico-ureteric reflux and other risk factors for renal damage: identification of high- and low-risk children. Acta Paediatr. 1999; 88: 31-9. (Ref. 77));
the CRP variable was dichotomized around the threshold corresponding to the median of the distribution of CRP values in patients without reflux, rounded to the next ten; this threshold is the one used previously in the literature: CRP greater than or equal to 40 mg/l: 1, CRP less than 40 mg/l: 0 (Gervaix A., Galetto-Lacour A., Gueron T., Vadas L., Zamora S., Suter S., Girardin E. Usefulness of procalcitonin and C-reactive protein rapid tests for the management of children with urinary tract infection. Pediatr. Infect. Dis. J. 2001; 20: 507-11. (Ref. 78); Galetto-Lacour A., Zamora S. A., Gervaix A. Bedside procalcitonin and C-reactive protein tests in children with fever without localizing signs of infection seen in a referral centre. Pediatrics 2003; 112: 1054-60. (Ref. 79); Tuerlinckx D., Vander Borght T., Glupczynski Y., Galanti L., Roelants V., Krug B., de Bilderling G., Bodart E. Is procalcitonin a good marker of renal lesions in febrile urinary tract infection? Eur. J. Pediatr. 2005; 164: 651-2. (Ref. 80); Prat C., Dominguez J., Rodrigo C., Gimenez M., Azuara M., Blanco S., Ausina V. Use of quantitative and semiquantitative procalcitonin measurements to identify children with sepsis and meningitis. Eur. J. Clin. Microbiol. Infect. Dis. 2004; 23: 136-8. (Ref. 81)).

### Statistical Analyses

First, the general characteristics of the population were described.

Secondly, the sample was dichotomized by a random stratified drawing (i.e. the patients were distributed into two groups at random, but respecting the percentage of high-grade vesicoureteral reflux in each group) for high-grade vesicoureteral reflux, into two-thirds for the construction of the clinical decision rule and one-third for its internal validation (arbitrary but usual proportion, Dubos F., Moulin F., Raymond J., Gendrel D., Breart G., Chalumeau M. Distinction des méningites bactériennes et virales de l′enfant: affinement d′une règle de décision clinique Arch. Pediatr. 2007; 14: 434-8. (Ref. 82); Chalumeau M, Chemaitilly W., Trivin C., Adan L., Breart G., Brauner R. Central precocious puberty in girls: an evidence-based diagnosis tree to predict central nervous System abnormalities. Pediatrics 2002; 109: 61-7. (Ref. 83)).

Thirdly, the distributions of the procalcitonin values were studied as a function of the presence or absence of high-grade vesicoureteral reflux. We compared them with a non-parameteric Mann-Whitney test.

Fourthly, the procalcitonin variable was dichotomized around different thresholds: 0.5 ng/ml (threshold chosen for previous publications (Leroy et al. Pediatrics, 2005, “Procalcitonin as a predictor of vesicoureteral reflux in children with a first febrile urinary tract infection” (Ref. 51), Leroy et al. Journal of Pediatrics, 2007, “Procalcitonin to reduce the number of unnecessary cystographies in children with a urinary tract infection: a European validation study”, and also chosen by other studies on the subject (Ref. 77 to 79, Bigot S., Leblond P., Foucher C., Hue V., D'Herbomez M., Foulard M. Apport du dosage de la procalcitonine pour le diagnostic de pyélonéphrite aiguë de l′enfant. Arch. Pediatr. 2005; 12: 1075-80. (Ref. 84); Gurgoze M. K., Akarsu S., Yilmaz E., Gödekmerdan A., Akça Z., Ciftçi I., Aygün A. D. Proinflammatory cytokines and procalcitonin in children with acute pyelonephritis. Pediatr. Nephrol. 2005; 20: 1445-8. (Ref. 85); Andreola B., Bressan S., Callegaro S., Liverani A., Plebani M., Da Dalt L. Procalcitonin and C-reactive protein as diagnostic markers of severe bacterial infections in febrile infants and children in the emergency department. Pediatr. Infect. Dis. J. 2007; 26: 672-7. (Ref. 87); Pecile P., Miorin E., Romanello C., Falleti E., Valent F., Giacomuzzi F., Tenore A. Procalcitonin: a marker of severity of acute pyelonephritis among children. Pediatrics 2004; 114: e249-54. (Ref. 88); Smolkin V., Koren A., Raz R., Colodner R., Sakran W., Halevy R. Procalcitonin as a marker of acute pyelonephritis in infants and children. Pediatr. Nephrol. 2002; 17: 409-12. (Ref. 89)), 0.6 ng/ml (Benador N., Siegrist C. A., Gendrel D., Greder C., Benador D., Assicot M., Bohuon C., Girardin E. Procalcitonin is a marker of severity of renal lesions in pyelonephritis. Pediatrics 1998; 102: 1422-5. (Ref. 90); Fernandez Lopez A., Luaces Cubells C., Garcia Garcia J. J., Fernandez Pou J. Procalcitonin in pediatric emergency departments for the early diagnosis of invasive bacterial infections in febrile infants: results of a multicenter study and utility of a rapid qualitative test for this marker. Pediatr. Infect. Dis. J. 2003; 22: 895-903. (Ref. 91)), 0.8 ng/ml ((Ref. 88)), 1 ng/ml (see, for example, (Ref. 87), (Ref. 85)), 1.5 ng/ml, 2 ng/ml (see (Ref. 78); (Ref. 79); (Ref. 86); (Ref. 87)), 2.5 ng/ml and 3 ng/ml.

For all of these thresholds, the relationship between high-grade vesicoureteral reflux and the dichotomized procalcitonin variable was studied by calculating the odds ratio (OR), the 95% confidence interval thereof and by the χ² test (see Ref. Huguier M., Flahault A. Biostatistiques au quotidien. Paris, France: Elsevier, 2003 (Ref. 101)) or Fisher (see Ref: Fisher R A. Statistical methods for research workers. 1934. Edition Oliver and Boyd, Edinburgh (Ref. 106)). In the case of a zero value in the contingency table, a corrected OR was calculated by adding, in each case of the table, the value 0.5 with respect to what is proposed by “Cochrane Collaboration” in the “Cochrane Reviewers' Handbook” (Ref. 93)

Fifthly, the relationship between high-grade vesicoureteral reflux and ureter dilation, pyelocaliceal cavities and renal size in the renal ultrasound was studied by calculation of the odds ratio (OR), the 95% confidence interval (CI) thereof and the χ² or Fisher test.

Sixthly, the presence of an interaction of the co-variables of interest on the relationship between high-grade vesicoureteral reflux and a high procalcitonin (at all of the thresholds defined above) and ureter dilation by the Breslow-Day test (see, for example, Greenland S. Tests for interaction in epidemiologic studies: a review and study of power. Stat Med 1983; 2: 243-251. (Ref. 110)) was sought.

Seventhly, a multivariate analysis by logistic regression (Hosmer, Lemeshow. Applied logistic regression. 2000 2^{nd} edition. Ed: Wiley-Interscience Publication, New York (Ref. 109)) made it possible to study the independence of the relationship between high-grade vesicoureteral reflux and a high procalcitonin, and each of the three ultrasound criteria, after adjustment for the co-variables of interest and for the centre (all of these co-variables were forced into the models). The relationship between high-grade vesicoureteral reflux and each of the ultrasound variables was not studied in the multivariate analysis (Hosmer, Lemeshow. Applied logistic regression. 2000 2^{nd} edition. Ed: Wiley-Interscience Publication, New York (Ref. 109)) unless this relationship was statistically significant in the univariate analysis (p<0.1—(Ref. 109)). A different model was constructed for each ultrasound variable (due to the presence of co-linearity) combined with each of the procalcitonin thresholds studied.

Eighthly, a clinical decision rule was constructed on the basis of the two predictors (high procalcitonin and the best ultrasound variable), which were combined by an “or”. A clinical decision rule was constructed for each procalcitonin threshold previously studied. We analyzed the relationship between high-grade vesicoureteral reflux and the clinical decision rules by the calculation of the OR and the 95% CI thereof. We then studied the discriminating power of each rule by calculating the sensitivity, specificity and 95% CI for the prediction of high-grade vesicoureteral reflux. For the implementation of the method of the invention, a specificity objective (corresponding to the number of normal cystographies or cystographies not showing correctly predicted low-grade vesicoureteral reflux, divided by the number of patients without high-grade vesicoureteral reflux) greater than 50%, a sensitivity constraint (corresponding to the number of cystographies showing correctly predicted high-grade vesicoureteral reflux, divided by the number of patients with high-grade vesicoureteral reflux) greater than 85% and the constraint of using an ultrasound criterion in order to take into account the clinicians' “beliefs” were fixed a priori and arbitrarily.

For the clinical decision rule(s) used, the positive and negative predictive values and the 95% CI thereof were calculated.

Ninthly, the method of the invention was validated on the remaining third of the population left under blind during the construction steps. No modification was made to the rule during its internal validation. A study on the discriminating performance by the calculations of sensitivity, specificity and 95% CI thereof for the prediction of high-grade vesicoureteral reflux was performed. Then, the results obtained with the detection method of the invention on the validation population were compared to those obtained on the construction population by means of a χ² test.

The patients with a semi-quantitative procalcitonin measurement (“PCT-Q”: results in the form of <0.5 ng/ml, 0.5-2 ng/ml or >2 ng/ml) were not included in the analyses regarding the procalcitonin variable considered to be a continuous variable, or in the analyses regarding the dichotomized procalcitonin variable when the threshold studied made this inclusion impossible (corresponding to the thresholds of: 0.6; 0.8; 1; 1.5; 2.5 and 3 ng/ml).

The software programs EPI INFO version 6.04 (Center of Disease Control and Prevention, Atlanta, Ga., USA) and Stata/SE 8 (StataCorp LP, College Station, Tex., USA) were used for the statistical analysis.

### Results

### Study Group

Data from seven centres (Afula, Israel (Ref. 90), Badalone, Spain (Ref. 82); Prat C., Dominguez J., Rodrigo C., Gimenez M., Azuara M., Jimenez O., Gali N., Ausina V. Elevated serum procalcitonin values correlate with renal scarring in children with urinary tract infection. Pediatr. Infect. Dis. J. 2003; 22: 438-42. (Ref. 92); Clamart, France (Gajdos V., Benattar C., Guérin S., Trioche P., Perreaux F., Mollet A., Foix L′Hélias L., Labrune P. Intérét de la procalcitonine et de la C-réactive protéine pour la prédiction des infections bactériennes sévères (IBS) chez les nourrissons fébriles âgés de moins de trois mois aux urgences. Congrès National de la Société de Pédiatrie, Lille, France, June 2004. Arch. Pediatr. 2004; 11: 728. (Ref. 94)), Geneva, Switzerland (see (Ref. 79), (Ref. 78)), Rzescow, Poland, (Korczowski B., Piasecka K., Duhl B., Bijos A. Procalcitonin and C-reactive protein as markers of bacteremia in children hospitalized with pneumonia and acute pyelonephritis. Pediatrica Polaska 2004; 129: 205-10. (Ref. 95), Udine, Italy (Ref. 88) and Yvoir, Belgium (Ref. 80) and that of the single-centre study (Ref. 52) were gathered. The patient characteristics for each centre are presented in Table 7. All of the centres were university hospitals (except for the hospital of Rzeszow, Poland, which was a general hospital), “tertiary” hospitals (except for the hospitals of Afula, Israel and Clamart, France, which were so-called “secondary” hospitals) and received children and adults (except for the hospitals of Geneva, Switzerland and Paris, France, which were exclusively paediatric hospitals)

Five hundred and ninety-five patients had the inclusion criteria. Contact was lost with forty-six patients (9%) before the cystography was performed. Five patients were not included in the analysis because they had undergone isotopic cystography, not enabling the analysis of vesicoureteral reflux by grade. For fifteen other patients (3%), the procalcitonin value at admission was not available. For thirteen patients (2%), the renal ultrasound was not performed. The analysis therefore related to 516 patients (87%).

The analysis of the continuous procalcitonin variable was performed on 494 patients (96%) because for 22 patients, the procalcitonin measurement was semi-quantitative. The analysis of the dichotomized procalcitonin variable was performed for all patients with a threshold of 0.5 and 2 ng/ml, for 509 patients (99%) with thresholds of 0.6, 0.8, 1 and 1.5 ng/ml and for 511 patients (99%) with thresholds of 2.5 and 3 ng/ml.

### Characteristics of the Population

The mean age of the entire population was 12.0 months (standard deviation=11.1 months; median=8.0 months; interquartile range=4.0-16). There were 208 boys (40%). Sixty-seven patients (14%) had a family history of uropathy. One hundred and twenty-five children (24%) had vesicoureteral reflux, and 56 patients (10%) had reflux of grade ≧3 (table 7).

TABLE 7
patient characteristics

| | | | | | |
|---|---|---|---|---|---|
| | | | Urinary | | |
| | | | collection modes | | |
| | | | (positivity | VUR | |
| | | | threshold for | of | |
| | | | bacteriuria used | any | |
| Center | | Inclusion | by | grade | VUR ≧3, |
| (“Centre”) | n (516) | dates | investigators)** | (%) | (%) |

Centers (centres) using SP or UC

| | | | | | |
|---|---|---|---|---|---|
| Afula | 56 | 1999-2000 | SP (10¹), UC | 25 | 11 |
| | | | (10³) | | |
| Badalona | 25 | 1998-2001 | SP (10³), UC | 20 | 20 |
| | | | (10⁴), MS (10⁵) | | |
| Geneva | 74 | 1998-2002 | SP (10³), UC | 30 | 14 |
| | | | (10⁴), MS (10⁵) | | |
| Udine | 80 | 2000-2002 | UC (5.10⁴), MS | 19 | 11 |
| | | | (10⁵) | | |
| Yvoir | 33 | 1999-2003 | SP (10³), UC | 21 | 12 |
| | | | (5.10⁴), MS | | |
| | | | (10⁵) | | |

Centres using CB

| | | | | | |
|---|---|---|---|---|---|
| Clamart | 23 | 2001-2002 | CB (10⁵) | 30 | 4 |
| Paris | 176 | 2001-2004 | CB (10⁵), MS | 25 | 9 |
| | | | (10⁵) | | |
| Rzeszow | 49 | 1997-1998, | CB (10⁵), MS | 22 | 8 |
| | | 2001-2004 | (10⁵) | | |
| Average | | | | 24 | 10 |

*Classified according to the urinary collection mode used for incontinent children.
**In units forming colonies/ml.
Abbreviations:
UC urethral catheter;
MS, urine collection in mid-stream;
SP, suprapubic puncture;
CB, collection bag;
VUR, vesicoureteral reflux.

### Development of the Method of the Invention

The construction of the decision rule for predicting high-grade vesicoureteral reflux used data from 344 patients, of whom 35 (10%) had high-grade vesicoureteral reflux. There were 141 boys (41%) and the mean age of the population was 12.0 months (standard deviation=11.3 months; median=8.0 months, interquartile range 4.0-16).

### Procalcitonin

In the analysis, as a continuous variable, the median procalcitonin of the patients with high-grade vesicoureteral reflux was significantly higher than that of patients without high-grade reflux: 3.3 by comparison with 0.7 ng/ml (p<0.001).

The procalcitonin variable was dichotomized at the different thresholds defined beforehand. There was a statistically significant association between high-grade vesicoureteral reflux and a high procalcitonin, regardless of the threshold chosen (Table 8). None of the co-variables studied interacted significantly with the relationship between high-grade vesicoureteral reflux and a high procalcitonin, regardless of the threshold considered (p>0.1).

A plurality of logistic regression models were constructed (one for each dichotomization threshold of the procalcitonin variable) with the data of 319 to 324 patients (91 to 93%), due to missing data on the procalcitonin variable (for the patients for whom the measurement was semi-quantitative—“PCT-Q)”). The association between vesicoureteral reflux and a high procalcitonin remained strong and significant regardless of the dichotomization threshold chosen (Table 8) after adjustment by logistic regression with respect to all of the co-variables of interest, ureter dilation in the renal ultrasound, and the centre.

TABLE 8
relationship between high-grade vesicoureteral reflux and high
procalcitonin as a function of the different thresholds chosen.

| | | | | | OR adjusted** | | OR adjusted | |
|---|---|---|---|---|---|---|---|---|
| | No VUR ≧ 3 | VUR ≧ 3 | OR [PCT] | | [PCT] | | [U dilation] | |
| PCT | n (%) | n (%) | (CI 95%) | p | (CI 95%) | p | (CI 95%) | p |
| ≧0.5 ng/ml | 187 (61) | 33 (96) | 11 (2.4-47) | <0.001 | 25 (3.2-194) | 0.002 | 5.2 (1.3-22) | 0.02 |
| <0.5 ng/ml | 122 (39) | 2 (4) | 1 | | 1 | | 1 | |
| ≧0.6 ng/ml | 170 (56) | 32 (91) | 8.4 (2.4-29) | <0.001 | 14 (3.1-64) | 0.001 | 6.1 (1.4-26) | 0.02 |
| <0.6 ng/ml | 134 (44) | 3 (9) | 1 | | 1 | | 1 | |
| ≧0.8 ng/ml | 145 (48) | 31 (89) | 8.5 (2.8-26) | <0.001 | 12 (3.5-44) | <0.001 | 6.4 (1.4-28) | 0.02 |
| <0.8 ng/ml | 159 (52) | 4 (11) | 1 | | 1 | | 1 | |
| ≧1 ng/ml | 136 (45) | 30 (86) | 7.4 (2.7-20) | <0.001 | 9.5 (3.1-29) | <0.001 | 6.1 (1.4-27) | 0.02 |
| <1 ng/ml | 168 (55) | 5 (14) | 1 | | 1 | | 1 | |
| ≧1.5 ng/ml | 109 (36) | 26 (74) | 5.2 (2.3-12) | <0.001 | 6.2 (2.6-15) | <0.001 | 7.9 (1.8-38) | 0.007 |
| <1.5 ng/ml | 195 (64) | 9 (26) | 1 | | 1 | | 1 | |
| ≧2 ng/ml | 89 (29) | 24 (69) | 5.4 (2.5-12) | <0.001 | 6.3 (2.7-15) | <0.001 | 7.5 (1.7-33) | 0.008 |
| <2 ng/ml | 220 (71) | 11 (31) | 1 | | 1 | | 1 | |
| ≧2.5 ng/ml | 78 (25) | 19 (56) | 3.7 (1.8-7.8) | <0.001 | 4.1 (1.9-9.2) | <0.001 | 6.8 (1.6-29) | 0.009 |
| <2.5 ng/ml | 228 (75) | 15 (44) | 1 | | 1 | | 1 | |
| ≧3 ng/ml | 63 (21) | 19 (56) | 4.9 (2.3-10) | <0.001 | 5.5 (2.4-12) | <0.001 | 7.0 (1.6-31) | 0.01 |
| <3 ng/ml | 243 (79) | 15 (44) | 1 | | 1 | | 1 | |

*The groups varied according to the thresholds, because for some patients the procalcitonin was measured by the semi-quantitative method. For these patients, the data was taken into account for thresholds 0.6 ng/ml, 0.8 ng/ml, 2.5 ng/ml and 3 ng/ml, OR adjusted in the logistic regression model on the co-variables of interest (sex, age, family history of uropathy, CPRP ≧ 40 mg/l) and the center(“centre”).
Abbreviations: CPC dilation, pyelocaliceal cavity dilation; U dilation, ureter dilation; CI, confidence interval; OR, odds ratio; PCT, procalcitonin; VUR, vesicoureteral reflux.

### Ultrasound Variables

The relationships between high-grade vesicoureteral reflux and the three ultrasound variables were measured by the OR as follows:
for ureter dilation: OR=3.2 (CI 95%=1.0-11; p=0.04);
for dilation of pyelocaliceal cavities: OR=2.4 (CI 95%=1.1-5.2; p=0.02);
for renal size: OR=1.1 (CI 95%=0.2-5.1; p=0.9).

The stratified analysis showed the presence of a statistically significant interaction (degree of significance of the Breslow-Day test=0.04) of the dilation of pyelocaliceal cavities on the relationship between high-grade vesicoureteral reflux and procalcitonin (regardless of the threshold considered). None of the other co-variables studied interacted statistically significantly on the relationship between high-grade vesicoureteral reflux and each of these two ultrasound variables (p>0.1). As the dilation of pyelocaliceal cavities interacted with the relationship between high-grade vesicoureteral reflux and a high procalcitonin, the multivariate analysis was therefore pursued only for ureter dilation. The association between high-grade vesicoureteral reflux and ureter dilation remained significant after adjustment of all of the co-variables of interest, a high procalcitonin (regardless of the dichotomization threshold considered) and the centre (Table 8).

Ureter dilation and a high procalcitonin, and dilation of the pyelocaliceal cavities and a high procalcitonin, were associated, constructing as many combinations as there were procalcitonin thresholds. The discriminating power of each of the predictors taken alone and that of each combination is presented in Table 9.

TABLE 9
Discriminating power of predictors taken alone
(regardless of the threshold chosen), then combined for
the population

| | Sensitivity (CI | | Specificity (CI | | | |
|---|---|---|---|---|---|---|
| Predictor(s) | 95%) | | 95%) | | OR (CI 95%) | |
| Ultrasound | | | | | | |
| variables | | | | | | |
| Ureter dilation | 11 | (5-26) | 96 | (93-98) | 3.2 | (1.0-11) |
| PCC dilation | 34 | (21-51) | 82 | (78-86) | 2.4 | (1.1-5.2) |
| Procalcitonin | | | | | | |
| PCT ≧0.5 ng/ml | 94 | (81-98) | 39 | (34-45) | 11 | (2.4-47) |
| PCT ≧0.6 ng/ml | 91 | (78-97) | 43 | (38-49) | 8.4 | (2.4-29) |
| PCT ≧0.8 ng/ml | 89 | (74-96) | 52 | (47-58) | 8.5 | (2.8-26) |
| PCT ≧1 ng/ml | 86 | (71-94) | 55 | (50-61) | 7.4 | (2.7-20) |
| PCT ≧1.5 ng/ml | 74 | (58-86) | 64 | (59-69) | 5.2 | (2.3-12) |
| PCT ≧2 ng/ml | 69 | (52-81) | 71 | (66-76) | 5.4 | (2.5-12) |
| PCT ≧2.5 ng/ml | 56 | (40-71) | 75 | (69-79) | 3.7 | (1.8-7.8) |
| PCT ≧3 ng/ml | 56 | (39-71) | 79 | (75-84) | 4.9 | (2.3-10) |

Combination of dilation of pyelocaliceal cavities and PCT

| | | | | | | |
|---|---|---|---|---|---|---|
| PCC or PCT | 100 | (90-100) | 31 | (26-36) | 32 | (2.0-529) |
| dilation ≧0.5 | | | | | | |
| PCC or PCT | 100 | (90-100) | 35 | (30-41) | 38 | (2.3-633) |
| dilation ≧0.6 | | | | | | |
| PCC or PCT | 100 | (90-100) | 43 | (37-48) | 53 | (3.2-869) |
| dilation ≧0.8 | | | | | | |
| PCC or PCT | 97 | (85-99) | 45 | (39-51) | 28 | (3.5-221) |
| dilation ≧1 | | | | | | |
| PCC or PCT | 91 | (78-97) | 52 | (47-58) | 12 | (3.4-41) |
| dilation ≧1.5 | | | | | | |
| PCC or PCT | 89 | (74-95) | 57 | (52-63) | 10 | (3.4-32) |
| dilation ≧2 | | | | | | |
| PCC or PCT | 82 | (66-92) | 60 | (55-65) | 7.0 | (2.7-18) |
| dilation ≧2.5 | | | | | | |
| PCC or PCT | 82 | (66-92) | 65 | (59-70) | 8.6 | (3.3-22) |
| dilation ≧3 | | | | | | |

Combination of ureter and PCT dilation

| | | | | | | |
|---|---|---|---|---|---|---|
| Ureter or PCT | 94 | (81-98) | 39 | (33-44) | 10 | (2.4-45) |
| dilation ≧0.5 | | | | | | |
| Ureter or PCT | 91 | (78-97) | 43 | (38-49) | 8.0 | (2.3-28) |
| dilation ≧0.6 | | | | | | |
| Ureter or PCT | 89 | (74-95) | 51 | (45-56) | 8.0 | (2.7-24) |
| dilation ≧0.8 | | | | | | |
| Ureter or PCT | 86 | (71-94) | 53 | (48-59) | 6.9 | (2.5-19) |
| dilation ≧1 | | | | | | |
| Ureter or PCT | 77 | (61-88) | 62 | (56-67) | 5.5 | (2.4-13) |
| dilation ≧1.5 | | | | | | |
| Ureter or PCT | 71 | (55-84) | 68 | (63-73) | 5.4 | (2.4-12) |
| dilation ≧2 | | | | | | |
| Ureter or PCT | 59 | (42-74) | 71 | (66-76) | 3.5 | (1.7-7.4) |
| dilation ≧2.5 | | | | | | |
| Ureter or PCT | 59 | (42-74) | 76 | (71-80) | 4.5 | (2.1-9.5) |
| dilation ≧3 | | | | | | |

Abbreviations:
PCC, pyelocaliceal cavities;
CI, confidence interval;
PCT, procalcitonin.

To improve the detection method, the combination of the measurement of procalcitonin with one of the following ultrasound criteria: ureter dilation (which was statistically associated with high-grade vesicoureteral reflux in the univariate and multivariate analysis), and dilation of pyelocaliceal cavities (which was statistically associated with high-grade reflux in the univariate analysis) was performed. All of the possible combinations were statistically associated with high-grade vesicoureteral reflux (Table 9).

### Study of the Efficacy of Detection Methods

Four combinations appeared to best satisfy the constraints set a priori (sensitivity ≧85% and specificity ≧50%): procalcitonin (≧1.8 or ≧2 ng/ml) combined by an “or” with dilation of the pyelocaliceal cavities, and procalcitonin (≧0.8 or 1 ng/ml) combined by an “or” with ureter dilation.

Arbitrarily, the “roundest” threshold for procalcitonin was chosen, for ease of memorization. The two most beneficial decision rules were thus: procalcitonin >1 ng/mL or the presence of ureter dilation on the renal ultrasound, and procalcitonin ≧2 ng/mL or the presence of dilation of the pyelocaliceal cavities on the renal ultrasound. In consideration of the higher frequency of the presence of dilation of the pyelocaliceal cavities as well as its more operator-dependent character with respect to the presence of ureter dilation, only the “procalcitonin ≧1 ng/mL or ureter dilation” model was preserved. More specifically, the detection of vesicoureteral reflux was evaluated with the combination of the following marker: a concentration of procalcitonin ≧1 ng/ml or the presence or ureter dilation on the renal ultrasound, positive and negative predictive values of 17% (CI 95%=13-24) and 97% (CI 95%=93-99), respectively.

The method overlooked five patients with high-grade vesicoureteral reflux (two grade-3 refluxes and three grade-4 refluxes). All were infants under one year of age. Two of them had undergone early renal DMSA scintigraphy: one was normal and the other showed grade-2 lesions according to the Benador classification (Ref. 90).

### Validation of the Detection Method of the Invention

The method of the invention was validated on the remaining third of the population left under blind, i.e. 172 patients, of whom 18 (10%) had high-grade vesicoureteral reflux. There were 67 boys (39%) and the mean age was 12.4 months (standard deviation=12.1 months; median=9.2 months, interquartile range=3.2-17).

The method of the invention combining the in vitro measurement of a serum concentration of procalcitonin greater than or equal to 1 ng/ml and the detection of ureter dilation is associated with high-grade vesicoureteral reflux in a statistically significant manner (OR=3.6; CI 95%=1.2-11; p=0.02). The results obtained with this method had a sensitivity of 72% (CI 95%=49-88) and a specificity of 58% (CI 95%=50-66) for the prediction of high-grade vesicoureteral reflux. There was no statistically significant difference between these sensitivities and specificities and those obtained for the construction population (p>0.1).

### Discussion

### Main Results

First, as presented in example 1, the ureter dilation was an ultrasound criterion correlated with vesicoureteral reflux.

There is a statistically significant and independent relationship between high-grade vesicoureteral reflux and ureter dilation on the renal ultrasound.

The method of the invention (presence of ureter dilation on the renal ultrasound or a serum concentration of procalcitonin greater than or equal to 1 ng/ml) makes it possible to predict high-grade vesicoureteral reflux with a sensitivity of around 80%. Its use may make it possible to avoid performing more than half of cystographies that are found to be unnecessary a posteriori because they are normal, while taking into account the clinicians' “beliefs”.

### Reproducibility of the Predictive Variables

The procalcitonin was measured according to validated and reproducible techniques (Ref. 98).

Comparison of the Method of the Invention with the Decision Rule Proposed by Oostenbrink et al.

A comparison of the method of the invention with the decision rule proposed by Oostenbrink et al.: Oostenbrink R., van der Heijden A. J., Moons K. G., Moll H. A. Prediction of vesico-ureteric reflux in childhood urinary tract infection: a multivariate approach. Acta Paediatr. 2000; 89: 806-10. (Ref. 96) was performed. To do this, the data from three centres (Paris, France, Udine, Italy (Ref. 88) and Yvoir, Belgium (Ref. 80), n=289), which made it possible to calculate the individual vesicoureteral reflux risk score defined by the Dutch team, were used. Among these patients, 27 (9%) had high-grade vesicoureteral reflux, 116 (40%) were boys and the mean age was 9.9 months (standard deviation=8.5 months; median=7.0 months, interquartile range=4.0-13). For this population, the association between high-grade vesicoureteral reflux and the method of the invention was at the significance limit (OR=2.2; CI 95%=0.9-5.2; p=0.054), while the score of Oostenbrink et al. was not statistically significantly associated with high-grade reflux (OR=2.7; CI 95%=0.6-17; p=0.3). The performance of the method of the invention was statistically different from that of the Oostenbrink score for predicting high-grade vesicoureteral reflux: the specificities were respectively 48% (CI 95%=42-54) and 18% (CI 95%=13-27) p<0.001; the sensitivities were respectively 70% (CI 95%=52-84%) and 93% (CI 95%=77-98) p=0.003. The threshold initially chosen by Oostenbrink et al. was then modified in order to increase the specificity of their prediction rule for high-grade vesicoureteral reflux (Table 11). For none of the other thresholds was there any statistically significant association between high-grade vesicoureteral reflux and the Oostenbrink risk score. The performances offered by the Oostenbrink score were calculated by modifying the statistical thresholds. By changing the threshold from 5 to 6, the specificity increased to 21% (CI 95%=17-26) but the sensitivity (81%; CI 95%=63-92) become lower than the constraint fixed a priori (85%). However, as the sensitivity of our decision rule for this population was also below 85%, we searched for the threshold of the score of Oostenbrink et al. offering the sensitivity closest to that of the first embodiment of the method of the invention, and we compared the specificities of the two clinical decision rules with this threshold. It is at the threshold of 8 that the sensitivity of the Oostenbrink rule was closest to that of the first embodiment of the method of the invention, even if it was slightly and statistically significantly higher: 74% (CI 95%=55-87) vs. 70% (CI 95%=52-84%); p=0.03. However, at this threshold, the specificity of the clinical decision rule of Oostenbrink et al. was statistically significantly below that of the first embodiment of the method of the invention: 27% (CI 95%=22-39) vs. 48% (CI 95%=42-54); p<0.001. Consequently, the rule of Oostenbrink et al. is less effective than that of the first embodiment of the method of the invention for this validation population, in spite of the attempt to modify the threshold of the risk score.

### Clinical Application

The replacement of the current strategy of systematically performing cystography with the method of the invention makes it possible to predict vesicoureteral reflux with a sensitivity of around 80% and a specificity of 50%. Among the five patients with high-grade vesicoureteral reflux overlooked by the clinical decision rule (including four grade-3 refluxes and one grade-4 reflux) and having undergone early renal scintigraphy, three had normal results, and the other two had low-grade renal lesions (1 and 2). Therefore, the method of the invention did not overlook patients with high-grade vesicoureteral reflux and high-grade scintigraphic renal lesions, while the prevalence of such lesions is 50% in the population of the three centres for which the information is available (Geneva, Switzerland ((Ref. 78), (Ref. 79) and (Ref. 90)), Udine, Italy (Ref. 88) and Yvoir, Belgium (Ref. 80)).

### Conclusion

The method of the invention therefore makes it possible to predict vesicoureteral reflux and therefore to reduce the number of unnecessary cystographies after a first febrile urinary tract infection in children. The prediction method of the invention provides a sensitivity close to 80% and enables around 55% of cystographies that are found to be unnecessary a posteriori to be avoided.

### Example 4

### Example of an Implementation of the Second Embodiment of the Method for Predicting High-Grade Vesicoureteral Reflux after a First Febrile Urinary Tract Infection in Children

### Patients and Methods

### General Description

As described in example 3, a secondary analysis of the data was performed with the single-centre cohort presented in Leroy et al. Pediatrics, “Procalcitonin as a predictor for vesico-ureteral reflux in children with urinary tract infection” (Ref. 14) and the multi-centre prospective data presented in Leroy et al. J. of Pediatrics, “Procalcitonin to reduce the number of unnecessary cystographies after a urinary tract infection in children: a European validation study” (Ref. 16) for the implementation of the second model for predicting vesicoureteral reflux. More specifically, this example relates to high-grade vesicoureteral reflux (defined as a grade greater than or equal to 3) after a first febrile urinary tract infection in children. The criteria for inclusion and non-inclusion are as follows: all of the patients were between 1 month and 4 years of age with a first febrile urinary tract infection defined by a rectal temperature greater than or equal to 38° C. associated with bacteriuria. Patients with known uropathy or having received an antibiotic treatment within the preceding 48 hours were not included. Children with an isotopic cystography (which did not enable high-grade vesicoureteral reflux to be distinguished) were not included. Patients having had a non-quantitative procalcitonin assay were not included.

As in example 3, the vesicoureteral reflux was identified on a radiological cystography by a senior radiologist with a blind for potential predictors and graded from 0 to 5 according to the international classification (Ref. 21). This predicted variable was then dichotomized as follows: reflux absent (grade 0) or low-grade (grades 1-2) by comparison with high-grade reflux, greater than or equal to 3).

### Predictive Variables

The serum procalcitonin of the patients was assayed prospectively by immunoluminometric analysis.

The other predictive variables were the same as in example 3, namely:
ureter dilation, pyelocaliceal cavity dilation and renal size. These ultrasound criteria were present on the ultrasound at the time of diagnosis of the urinary tract infection by a senior paediatric radiologist, with a blind for the result of the procalcitonin assay.
presence of family history of first-degree uropathy. Variables dichotomized as follows: presence of family history: 1, absence: 0 (Feather S. Vesicoureteric reflux: all in the genes? Lancet 1996; 348: 725-8. (Ref. 73); Chertin B., Puri P. Familial vesicoureteral reflux. J. Urol. 2003; 169: 1804-8. (Ref. 74));
sex of the patient. Variable dichotomized as follows: boy: 1, girl: 0 (Jakobsson B., Jacobson S. H., Hjalmas K. Vesico-ureteric reflux and other risk factors for renal damage: identification of high- and low-risk children. Acta Paediatr. 1999; 88: 31-9. (Ref. 77));
age less than or equal to 1 year: 1, age greater than 1 year: 0 (Jacobson S. H., Hansson S., Jakobsson B. Vesico-ureteric reflux: occurrence and long-term risks. Acta Paediatr. 1999; 88: 22-30. (Ref. 75); Gelfand M. J., Koch B. L., Cordero G. G., Salmanzadeh A., Gartside P. S. Vesicoureteral reflux: subpopulations of patients defined by clinical variables. Pediatr. Radiol. 2000; 30: 121-4. (Ref. 76));
the C-Reactive protein (CRP) at the time of diagnosis of the urinary tract infect. This variable was taken into consideration as a continuous variable in the analysis.

### Statistical Analyses

First, the general characteristics of the population were studied.

Secondly, an analysis of the relationship between high-grade vesicoureteral reflux and the predictive variables by a univariate analysis using a logistic regression model was performed. For the continuous variables (CRP and PCT), the linearity of the relationship between the logit (defined as in ref.: Hosmer, Lemeshow. Applied logistic regression. 2000 2^{nd} edition. Ed: Wiley-Interscience Publication, New York (Ref. 109)) and the probability of high-grade vesicoureteral reflux and the continuous variable was tested, according to the recommendation of Royston, Sauerbrei. Multivariable model building. A pragmatic approach to regression analysis based on fractional polynomials for modelling continuous variables. 2008. Ed: Wiley and Sons Ltd., Chichester, England (Ref. 108). In the case of a relationship statistically different from linearity, the continuous variable was transformed into a fractional polynomial of which the coefficients and the degree were defined by making a compromise between the maximization of the likelihood of the logistic regression model and the simplicity of the mathematical equation obtained (Ref. 108). Only the variables associated with high-grade vesicoureteral reflux with a degree of significance lower than 0.1 were integrated in the multivariate logistic regression model descending step-by-step (Ref. 109). The appropriateness of the model was measured by the Hosmer Lemeshow coefficient (Ref. 109). The co-linearity (Ref. 109) was tested between the ultrasound variables by a logistic regression model, and between the inflammatory markers (CRP and PCT) by a linear regression model (defined by the reference: Armitage, Barry, Matthews. Statistical methods in medical research. 4th edition. 2002. Ed: Blackwell Science Ltd., Berlin, Germany (Ref. 111)). For the binary variables, in the case of co-linearity, the addition to the model of the variable least strongly associated with high-grade vesicoureteral reflux was evaluated: if this addition did not statistically significantly improve the likelihood, it was not kept in the multivariate model. For the continuous variables, in the case of co-linearity, the areas below the ROC curve of the variables were calculated. Only the variable with the largest area below the ROC curve was kept for the construction of the multivariate model. The discriminating power of the final multivariate logistic regression model was evaluated by producing a ROC curve and calculating its area below the curve accompanied by its confidence interval (CI 95%).

### Results

### Study Groups

Data from seven center (“centres”) (Afula, Israel (Ref. 90), Badalone, Spain (Ref. 82); Prat C., Dominguez J., Rodrigo C., Gimenez M., Azuara M., Jimenez 0., Gali N., Ausina V. Elevated serum procalcitonin values correlate with renal scarring in children with urinary tract infection. Pediatr. Infect. Dis. J. 2003; 22: 438-42. (Ref. 92); Clamart, France (Gajdos V., Benattar C., Guérin S., Trioche P., Perreaux F., Mollet A., Foix L′Hélias L., Labrune P. Intérât de la procalcitonine et de la C-réactive protéine pour la prédiction des infections bactériennes sévéres (IBS) chez les nourrissons fébriles ágés de moins de trois mois aux urgences. Congrès National de la Société de Pédiatrie, Lille, France, June 2004. Arch. Pediatr. 2004; 11: 728. (Ref. 94)), Geneva, Switzerland (see (Ref. 79), (Ref. 78)), Rzescow, Poland, (Korczowski B., Piasecka K., Duhl B., Bijos A. Procalcitonin and C-reactive protein as markers of bacteriemia in children hospitalized with pneumonia and acute pyelonephritis. Pediatrica Polaska 2004; 129: 205-10. (Ref. 95), Udine, Italy (Ref. 88) and Yvoir, Belgium (Ref. 80) and those of the single-centre study (Ref. 51) were gathered. The patient characteristics for each centre are presented in Table 7, appearing in example 3. All of the centres were university hospitals (except for the hospital of Rzeszow, Poland, which was a general hospital), “tertiary” hospitals (except for the hospitals of Afula, Israel and Clamart, France, which were so-called “secondary” hospitals) and received children and adults (except for the hospitals of Geneva, Switzerland and Paris, France, which were exclusively paediatric hospitals)

Five hundred and ninety-five patients had the inclusion criteria. Contact was lost with forty-six patients (9%) before the cystography was performed. Five patients were not included in the analysis because they had undergone isotopic cystography, not enabling the analysis of vesicoureteral reflux by grade. For fifteen other patients (3%), the procalcitonin value at admission was not available. For 22 patients (4%), the procalcitonin value was semi-quantitative. For thirteen patients (2%), the renal ultrasound was not performed. The analysis therefore related to 494 patients (83%).

### Characteristics of the Population

The mean age of the entire population was 12.1 months (standard deviation=11.2 months; median=8.0 months; interquartile range=4.0-17). There were 198 boys (40%). Sixty-four patients (14%) had a family history of uropathy. One hundred and twenty-five children (25%) had vesicoureteral reflux, and 56 patients (11%) had high-grade reflux.

### Development of the Method for Prediction of High-Grade Vesicoureteral Reflux

The relationships between high-grade vesicoureteral reflux and the binary predictors are presented in Table 10. Only dilation and dilation of pyelocaliceal cavities were associated with high-grade vesicoureteral reflux with a degree of significance lower than 0.1. In consideration of the strong co-linearity of these two variables, the inventors integrated only one of these two variables in the multivariate model. Ureter dilation was chosen because its odds ratio was higher, its frequency lower, and also because this variable was identified as being the best predictor in the previous meta-analysis (example 1) and due to its lower operator-dependent character. Finally, the addition of the pyelocaliceal cavity dilation variable to the multivariate model did not statistically significantly improve the likelihood of the prediction method.

TABLE 10
Relationship between high-grade
vesicoureteral reflux and the different predictors.

| | | | adjusted OR | |
|---|---|---|---|---|
| Variables | OR (CI 95%) | p | (CI 95%) | P |
| Sex | 0.8 (0.5-1.5) | 0.8 | — | — |
| History of | 0.9 (0.4-2.1) | 0.8 | — | — |
| uropathy** | | | | |
| Age ≦1 | 1.1 (0.6-2.0) | 0.7 | | |
| year | | | | |
| PCC | 2.5 (1.4-4.6) | 0.002 | —§ | — |
| dilation | | | | |
| Ureter | 5.7 (2.5-13.4) | <0.001 | 9.5 (3.1-29) | <0.001 |
| dilation | | | | |
| Renal size | 1.6 (0.6-4.0) | 1.0 | — | — |

*OR adjusted in the final logistic regression model,
**Group n = 467 for this variable due to missing data,
§Variable not taken into consideration due to the co-linearity between this variable and ureter dilation;
Abbreviation:
PCC pyelocaliceal cavities.

The relationship between the probability of high-grade vesicoureteral reflux and procalcitonin as a quantitative variable was statistically different from the linearity (p<0.001). This variable was therefore transformed in the form of a first-degree fractional polynomial, in the following form: Pct-transf=(Pct/100)^{Λ}(−0.5)−4.89. The procalcitonin thus transformed was significantly associated with high-grade vesicoureteral reflux (p<0.001). The likelihoods of the first- and second-degree fractional polynomials transforming the procalcitonin variable were not significantly different. Consequently, the inventors chose the first-degree fractional polynomial, corresponding to the simplest mathematical equation. There is a statistically significant association between high-grade vesicoureteral reflux and CRP (p<0.001). It was taken into account in the logistic regression equation in linear form because its relationship with the logit of high-grade vesicoureteral reflux probability was not statistically different from the linearity. There was co-linearity between the procalcitonin variables and CRP, of which the areas below the ROC curve were respectively 0.72 and 0.59 (see FIG. 3 p<0.01). Consequently, only the procalcitonin variable was kept in the multivariate model.

The following variables: procalcitonin transformed in the form of a first-degree fractional polynomial and ureter dilation were chosen. These two variables remained statistically significantly associated with high-grade vesicoureteral reflux (p<0.01) in the multivariate model (p<0.001). The Hosmer-Lemeshow coefficient was 0.85, the area below the ROC curve of the model was 0.75.

The method of the invention therefore makes it possible to perform a cystography in the only patients with a result of the mathematical equation (obtained from the logistic regression model) calculated as follows:

Vr=1/[1+exp(−a*ureter dilatation−b*[(Pct/100)^{(c)}+d]−e)]

is greater than or equal to 0.072.

Thus, the detection method of the invention has a sensitivity of at least 85%, and a specificity of 47% for the prediction of high-grade vesicoureteral reflux and thus makes it possible not to overlook more than 5% of children with high-grade vesicoureteral reflux.

### Example 5

### Example of Execution of the First Embodiment of the Method of the Invention

A blood sample of 0.5 ml was taken from a patient with a first febrile urinary tract infection. The first febrile urinary tract infection was diagnosed by the presence of a rectal temperature above 38° C. and bacteriuria (quantified at 10⁵ colonies forming one unit/mL (Colonies Forming Units/mL) of a single pathogenic germ on a urine sample collected by a sterile collection bag). The measurement of the procalcitonin concentration in the sample taken was performed by an immunoluminometric technique using the PCT LUMItest kit (BRAHMS, Hennigsdorf, Germany). The concentration measured was greater than or equal to 1 ng/mL. A procalcitonin value of 1 was therefore assigned to this patient.

An ultrasound image of the urinary system was also produced for the patient with a PHILIPS HDI 500 ultrasound apparatus. A measurement of the cross-section of the ureter was performed on the ultrasound image obtained; it had a cross-section of more than one millimetre in diameter, corresponding to dilation of the ureter. An ultrasound value of 1 was therefore assigned to this patient.

The sum of the procalcitonin and ultrasound values was obtained, and was 2. The patient was therefore considered to be at high risk for vesicoureteral reflux. This model had a sensitivity of 86% and a specificity of 53%.

A blood sample on another patient with the same clinical signs was obtained and the measurement of the procalcitonin concentration was performed as described above. The measurement of the procalcitonin concentration was strictly less than 1 ng/mL, and the procalcitonin value was therefore equal to 0. An ultrasound image of the urinary system was also obtained for the patient as described above. A measurement of the cross-section of the ureter was performed on the ultrasound image obtained; it had a cross-section of less than one millimetre and did not therefore correspond with a dilation of the ureter. An ultrasound value of 0 was therefore assigned to this patient.

The sum of the procalcitonin and ultrasound values was obtained, and was 0. The patient was therefore considered to be at low risk for high-grade vesicoureteral reflux, with a probability of not overlooking such a reflux of 97%.

As shown in this example, the method of the invention also makes it possible to predict vesicoureteral reflux and avoid the production of unnecessary vesicoureteral cystography.

### Example 6

### Example of Execution of the First Embodiment of the Method of the Invention

A blood sample of 0.5 ml was taken from a patient with a first febrile urinary tract infection. The first febrile urinary tract infection was diagnosed by the presence of a rectal temperature above 38° C. and bacteriuria (quantified at 10⁵ colonies forming one unit/mL (Colonies Forming Units/mL) of a single pathogenic germ on a urine sample collected by a sterile collection bag). The measurement of the procalcitonin concentration in the sample taken was performed by an immunoluminometric technique using the PCT LUMItest kit (BRAHMS, Hennigsdorf, Germany). The concentration measured was equal to 1.2 ng/mL.

An ultrasound image of the urinary system was also produced for the patient with a PHILIPS HDI 500 ultrasound apparatus. A measurement of the cross-section of the ureter was performed on the ultrasound image obtained; it had a cross-section of more than one millimetre in diameter, corresponding to dilation of the ureter. An ultrasound value of 1 was therefore assigned to this patient.

The procalcitonin concentration measured and the ultrasound value for the dilation of the ureter were combined mathematically by means of the following mathematical equation:

Vr=1/[1+exp(−a*ureteral dilation−b*[(Pct/100)^{(c)}+d]−e)]

in which a, b, c, d and e were coefficients obtained from the logistic regression model with the following values: −1.53564 for a, −0.129002 for b, −0.5 for c, −4.887527991 for d and −1.553866 for e. The result of this equation was 0.3623112. As this result is greater than the defined threshold of 0.072, the patient was considered to belong to the group at high risk for high-grade vesicoureteral reflux. This model provides a sensitivity of 86% and a specificity of 47% for the prediction of high-grade vesicoureteral reflux.

A blood sample from another patient with the same clinical signs was obtained and the measurement of the procalcitonin concentration was performed as described above. The measurement of the procalcitonin concentration was 0.3. An ultrasound image of the urinary system was also produced for the patient as described above. A measurement of the cross-section of the ureter was performed on the ultrasound image obtained, and it had a cross-section of less than one millimetre and did not therefore correspond to a dilation of the ureter. An ultrasound value of 0 was therefore assigned to this patient. The two values are combined by means of the same mathematical equation as described above, which gives the same result of 0.03631331. This result was lower than the defined threshold of 0.072, and the patient was therefore considered to belong to the group at low risk for high-grade vesicoureteral reflux, with a likelihood of overlooking high-grade vesicoureteral reflux of 95%. This model has a sensitivity of 86% and a specificity of 47% for the prediction of high-grade vesicoureteral reflux.

As demonstrated in this example, the method of the invention also makes it possible to predict vesicoureteral reflux and avoid performing unnecessary vesicoureteral cystographies.

### LIST OF REFERENCES

(Ref. 1) Hoberman A., Chao H. P., Keller D. M., Hickey R., Davis H. W., Ellis D. Prevalence of urinary tract infection in febrile infants. J. Pediatr. 1993; 123: 17-23.
(Ref. 2) M\dot{a}rild S., Jodal U. Incidence rate of first-time symptomatic urinary tract infection in children under 6 years of age. Acta Paediatr. 1998; 87: 549-52.
(Ref. 3) Jacobson S. H., Hansson S., Jakobsson B. Vesico-ureteric reflux: occurrence and long-term risks. Acta Paediatr Suppl. 1999; 88: 22-30.
(Ref. 4) Guillot M., Eckart P., Dacher J. N. Initial imaging in pediatric urinary tract infection.

Arch Pediatr. 1998; 5 Suppl: 282S-284S.
(Ref. 5) American Academy of Pediatrics, Committee on Quality Improvement, Subcommittee on Urinary Tract Infection. Practice parameter: the diagnosis, treatment, and evaluation of the initial urinary tract infection in febrile infants and young children. Pediatrics 1999; 103: 843-52.
(Ref. 6) Fotakis M., Molyvda Athanasopoulou E., Psarrakos K., Economou I. Radiation doses to paediatric patients up to 5 years of age undergoing micturating cystourethrography examinations and its dependence on patient age: a Monte Carlo study. Br. J. Radiol. 2003; 76: 812-7.
(Ref. 7) Guignard J. P. Urinary infection after micturating cystography. Lancet. 1979; 1: 103.
(Ref. 8) Hägglöf B. Psychological reaction by children of various ages to hospital care and invasive procedures. Acta Paediatr Suppl. 1999; 88: 72-8.
(Ref. 9) Nicklasson L., Hög\dot{a}rd S. Cost-analysis of management strategies for children with vesico-ureteric reflux. Acta Paediatr Suppl. 1999; 88: 79-86.
(Ref. 10) Chalumeau et al. “Procalcitonine et prédiction dureflux vésico-urétéral après une première infection urinaire fébrile chez l′enfant” Journée Parisienne de pédiatrie. Flammarion Médecine-Sciences, Paris 2005: 155-62.
(Ref. 11) Westwood M. E., Whiting P. F., Cooper J., Watt I. S., Kieijnen J. Further investigation of confirmed urinary tract infection (UTI) in children under five years: a systematic review. BMC Pediatr. 2005; 5: 2
(Ref. 12) Cucherat M. Méta-analyse des essais therapeutiques. Paris, France: Masson, 1997
(Ref. 13) Hoberman A., Charron M., Hickey R. W., Baskin M., Kearney D. H., Wald E. R. Imaging studies after a first febrile urinary tract infection in young children. N. Engl. J. Med. 2003; 348: 195-202.
(Ref. 14) Leroy et al. “Procalcitonin as a predictor for vesico-ureteral reflux in children with urinary tract infection.” (Pediatrics, June 2005; 115: e706-709).
(Ref. 15) Wasson J. H., Sox H. C., Neff R. K., Goldman L. Clinical prediction rules. Applications and methodological standards. N. Engl. J. Med. 1985; 313: 793-9
(Ref. 16) Leroy et al. “Procalcitonin to reduce the number of unnecessary cystographies after a urinary tract infection in children: a European validation study.” (Journal of Pediatrics, January 2007; 150: 89-95)
(Ref. 17) Oostenbrink et al. “Prediction of vesicoureteric reflux in childhood urinary tract infection: a multivariative approach (Acta Paediatr 2000; 89: 806-10)
(Ref. 18) Leroy et al., “Prediction of vesicoureteral reflux after a first febrile urinary tract infection in children: validation of a clinical decision rule.” (Archives of Disease in Childhood, March 2006; 91; 241-244)
(Ref. 19) Royton P., Sauerbrei W. Multivariable model-building Ed. Wiley, 2008
(Ref. 20) Downs S M. Technical report: urinary tract infections in febrile infants and young children. The Urinary Tract Subcommittee of the American Academy of Pediatrics Committee on Quality Improvement. Pediatrics. 1999; 103: e54
(Ref. 21) Lebowitz R. L., Olbing H., Parkkulainen K. V., Smellie J. M., Tamminen-Mobius T. E. International System of radiographic grading of vesicoureteric reflux. International Reflux Study in Children. Pediatr. Radiol. 1985; 15: 105-9.).
(Ref. 22) Westwood M. E., Whiting P. F., Cooper J., Watt I. S., Kleijnen J. Further investigation of confirmed urinary tract infection (UTI) in children under five years: a systematic review. BMC Pediatr. 2005; 5: 2.
(Ref. 23) DerSimonian R. L., N. Meta-analysis in clinical trials. Control Clin. Trials 1986; 7: 177-88.
(Ref. 24) Deeks J. J., Higgins J. P. T., Altman D. G., editors. Analysing and presenting results. In: Alderson P, Green S, Higgins J, editors. Cochrane Reviewers' Handbook 4.2.2 [updated November 2004]; Section 8.
(Ref. 25) Dura Trave T., Gonzalez Montera R., Juste Ruys M., Gonzales de Dios F., Carratala Marco M., Moya Benavent M., Verdu Rico J., Caballero Calpena O. Utilidad de la gammagrafia rénal en la valoracion de la primera infeccion febril en la edad pediatrica. An Esp Pediatr. 1997; 47: 378-82.
(Ref. 26) Evans E. D., Meyer J. S., Harty M. P., Bellah R. D. Assessment of increase in renal pelvic size on post-void sonography as a predictor of vesicoureteral reflux. Pediatr. Radiol. 1999; 29: 291-4.
(Ref. 27) Kenney I. J., Negus A. S., Miller F. N. Is sonographically demonstrated mild distal ureteric dilatation predictive of vesicoureteric reflux as seen on micturating cystourethrography? Pediatr. Radiol. 2002; 32: 175-8.
(Ref. 28) Muensterer O. J. Comprehensive ultrasound versus voiding cysturethrography in the diagnosis of vesicoureteral reflux. Eur. J. Pediatr. 2002; 161: 435-7.
(Ref. 29) Peratoner L., Pennesi M., Bordugo A., Melega R., Sorce P., Travan L., Minisini S., Zennaro F., Da Ronch L. Kidney length and scarring in children with urinary tract infection: importance of ultrasound scans. Abdom. Imaging 2005; 30: 780-5.
(Ref. 30) Tsai Y. C., Hsu C Y, Lin G. J., Wang C. J., Cheng C. H., Huang Y H, Yen M. H., Hsia S. H., Yan D. C. Vesicoureteral reflux in hospitalized children with urinary tract infection: the clinical value of pelvic ectasia on renal ultrasound, inflammatory responses and demographic data. Chang Gung Med. J. 2004; 27: 436-42.
(Ref. 31) Egger M., Smith G. D., Altman D. G. Systematic reviews in health care: meta-analysis in context. London, United Kingdom: BMJ Publishing Group, 2001.
(Ref. 32) Lijmer J. G., Bossuyt P. M., Heisterkamp S. H. Exploring sources of heterogeneity in systematic reviews of diagnostic tests. Stat. Med. 2002; 21: 1525-37.
(Ref. 33) Moses L., Littenberg B., Shapiro D. Combining independent studies of a diagnostic test into a summary ROC curve: data-analytical approaches and some additional consideration. Stat. Med. 1993; 12: 1293-316.
(Ref. 34) Zamoura J., Abraira V., Muriel A., Khan K., Coomarasamy A. Meta-Disc: a software for meta-analysis of test accuracy data. BMC Med. Res. Methodol. 2006; 6: 1-12.
(Ref. 35) Gordon I., Barkovics M., Pindoria S., Cole T. J., Woolf A. S. Primary vesicoureteric reflux as a predictor of renal damage in children hospitalized with urinary tract infection: a systematic review and meta-analysis. J. Am. Soc. Nephrol. 2003; 14: 739-44.
(Ref. 36) Egger M., Davey Smith G., Schneider M., Minder C. Bias in meta-analysis detected by a simple, graphical test. BMJ 1997; 315: 629-34.
(Ref. 37) Hoberman A., Charron M., Hickey R. W., Baskin M., Kearney D. H., Wald E. R. Imaging studies after a first febrile urinary tract infection in young children. N. Engl. J. Med. 2003; 348: 195-202.
(Ref. 38) Almeida H. N., Ribeiro M., Colarinha J., Santos J. F., Rosa F. C. Imaging methods in the study of urinary tract infections in children. Acta Med Port 1994; 7: 15-20.
(Ref. 39) Alon U., Pery M., Davidai G., Berant M. Ultrasonography in the radiologic evaluation of children with urinary tract infection. Pediatrics 1986; 78: 58-64.
(Ref. 40) Alon U.S., Ganaphthy S. Should renal ultrasonography be done routinely in children with first urinary tract infection? Clin. Pediatr. (Phila) 1999; 38: 21-5.
(Ref. 41) Baronciani D., Bonora G., Andreola A., Cambie M., Nedbal M., dell'Agnola C. A. Ruolo dell'ecografia dell'iter diagnostico delle infezioni delle vie urinarie. Riv Ital Ped 1986; 12: 214-20.
(Ref. 42) Boudailliez B., McMahon Y., Grumbach Y., Baratte B., Caraco M. H., Piussan C. Place de l′échographie dans l′investigation: une première infection urinaire chez l′enfant. Arch Pediatr 1989; 46: 113-6.
(Ref. 43) Calisti A., Perrotta M. L., Oriolo L., Ingianna D., Sciortino R. Diagnostic workup of urinary tract infections within the first 24 months of life, in the era of prenatal diagnosis. The contribution of different imaging techniques to clinical management. Minerva Pediatr. 2005; 57: 269-73.
(Ref. 44) Cleper R., Krause I., Eisenstein B., Davidovits M. Prevalence of vesicoureteral reflux in neonatal urinary tract infection. Clin. Pediatr. (Phila) 2004; 43: 619-25.
(Ref. 45) Davey M. S., Zerin J. M., Reilly C., Ambrosius W. T. Mild renal pelvic dilatation is not predictive of vesicoureteral reflux in children. Pediatr. Radiol. 1997; 27: 908-11.
(Ref. 46) David C., Dacher J. N., Monroc M., Eurin D., Le Dosseur P. Retrograde cystography after a first episode of acute pyelonephritis in the child and adolescent. J. Radiol. 1998; 79: 133-7.
(Ref. 47) DiPietro M. A., Blane C. E., Zerin J. M. Vesicoureteral reflux in older children: concordance of US and voiding cystourethrographic findings. Radiology 1997; 205: 821-2.
(Ref. 48) Foresman W. H., Hulbert W. C., Rabinowitz R. Does urinary tract ultrasonography at hospitalization for acute pyelonephritis predict vesicoureteral reflux? J. Urol. 2001; 165: 2232-4.
(Ref. 49) Gelfand M. J., Barr L. L., Abunku O. The initial renal ultrasound examination in children with urinary tract infection: the prevalence of dilated uropathy has decreased. Pediatr. Radiol. 2000; 30: 665-70.
(Ref. 50) Goldman M., Lahat E., Strauss S., Reisler G., Livne A., Gordin L., Aladjem M. Imaging after urinary tract infection in male neonates. Pediatrics 2000; 105: 1232-5.
(Ref. 51) Honkinen O., Ruuskanen O., Rikalainen H., Makinen E. O., Valimaki I. Ultrasonography as a screening procedure in children with urinary tract infection. Pediatr. Infect. Dis. J. 1986; 5: 633-5.
(Ref. 52) Leroy S., Adamsbaum C., Marc E., Moulin F., Raymond J., Gendrel D., Breart G., Chalumeau M. Procalcitonin as a predictor of vesicoureteral reflux in children with a first febrile urinary tract infection. Pediatrics 2005; 115: e706-9.
(Ref. 53) Leroy S., Romanello C., Galetto-Lacour A., Smolkin V., Korczowski B., Rodrigo C., Tuerlinckx D., Gajdos V., Moulin F., Contardo M., Gervaix A., Halevy R., Duhl B., Prat C., Borght T. V., Foix-l'Helias L., Dubos F., Gendrel D., Breart G., Chalumeau M., Marc E., Adamsbaum C. “Procalcitonin to reduce the number of unnecessary cystographies in children with a urinary tract infection: a European validation study”. J. Pediatr. 2007; 150: 89-95.
(Ref. 54) Mage K., Zoppardo P., Cohen R., Reinert P., Ponet M. Imaging and the first urinary infection in children. Respective role of each test during the initial evaluation a propos of 122 cases. J. Radiol. 1989; 70: 279-83.
(Ref. 55) Mahant S., Friedman J., MacArthur C. Renal ultrasound findings and vesicoureteral reflux in children hospitalised with urinary tract infection. Arch. Dis. Child. 2002; 86: 419-20.
(Ref. 56) Morin D., Veyrac C., Kotzki P O., Lopez C., Dalla Vale F., Durand M F., Astruc J., Dumas R. Comparison of ultrasound and dimercaptosuccinic acid scintigraphy changes in acute pyelonephritis. Pediatr. Nephrol. 1999; 13: 219-22.
(Ref. 57) Oostenbrink R., van der Heijden A. J., Moons K. G., Moll H. A. Prediction of vesico-ureteric reflux in childhood urinary tract infection: a multivariate approach. Acta Paediatr. 2000; 89: 806-10.
(Ref. 58) Smellie J. M., Rigden S. P., Prescod N. P. Urinary tract infection: a comparison of four methods of investigation. Arch. Dis. Child. 1995; 72.
(Ref. 59) Tan S. M., Chee T., Tan K. P., Cheng H. K., Ooi B. C. Role of renal ultrasonography (RUS) and micturating cystourethrogram (MCU) in the assessment of vesico-ureteric reflux (VUR) in children and infants with urinary tract infection (UTI). Singapore Med. J. 1988; 29: 150-2.
(Ref. 60) Taskinen S., Ronnholm K. Post-pyelonephritic renal scars are not associated with vesicoureteral reflux in children. J. Urol. 2005; 173: 1345-8.
(Ref. 61) Yen C. W., Chen D. H. Urinary tract infection in children. J. Microbiol. Immunol. Infect. 1999; 32: 199-205.
(Ref. 62) Zamir G., Sakran W., Horowitz Y., Koren A., Miron D. Urinary tract infection: is there a need for routine renal ultrasonography? Arch. Dis. Child. 2004; 89: 466-8.
(Ref. 63) Zocchi M., Ferrera C. G. Importance of echography in the study of urinary tract infections in childhood. Radio'. Med. 1988; 76: 597-600.
(Ref. 64) Baert A. L., Sartor K. Pediatric uroradiology. Heidelberg, Germany: Springer, 2001.
(Ref. 65) Chalumeau M., Leroy S., Gendrel D., Bréart G. Procalcitonine et prédiction dureflux vésico-urétéral après une première infection urinaire febrile chez l′enfant. Journées Parisiennes de Pédiatrie. Flammarion Médecine-Sciences, Paris 2005: 155-62.
(Ref. 66) Lijmer J. G., Mol B. W., Heisterkamp S., Bonsel G. J., Prins M. H., van der Meulen J. H., Bossuyt P. M. Empirical evidence of design-related bias in studies of diagnostic tests. JAMA 1999; 282: 1061-6.
(Ref. 67) Whiting P., Rutjes A. W., Reitsma J. B., Bossuyt P. M., Kleijnen J. The development of QUADAS: a tool for the quality assessment of studies of diagnostic accuracy included in systematic reviews. BMC Med. Res. Methodol. 2003; 3: 1-13.
(Ref. 68) Roy P. M., Colombet I., Durieux P., Chatellier G., Sors H., Meyer G. Systematic review and meta-analysis of strategies for the diagnosis of suspected pulmonary embolism. BMJ 2005; 331: 259.
(Ref. 69) Doria A. S., Moineddin R., Kellenberger C. J., Epelman M., Beyene J., Schuh S., Babyn P. S., Dick P. T. US or CT for Diagnosis of Appendicitis in Children and Adults? A Meta-Analysis. Radiology 2006; 241: 83-94.
(Ref. 70) Jadad A. R., Moore R. A., Carroll D., Jenkinson C., Reynolds D. J., Gavaghan D. J., McQuay H. J. Assessing the quality of reports of randomized clinical trials: is blinding necessary? Control Clin. Trials 1996; 17: 1-12.
(Ref. 71) Wasson J. H., Sox H. C., Neff R. K., Goldman L. Clinical prediction rules. Applications and methodological standards. N. Engl. J. Med. 1985; 313: 793-9.
(Ref. 72) Michael Meisner “Procalcitonin, a new, innovative infection parameter. Biochemical and clinical aspects”, 3rd revised and expanded edition, Stuttgart, Germany, Thieme, 2000
(Ref. 73) Feather S. Vesicoureteric reflux: all in the genes? Lancet 1996; 348: 725-8.
(Ref. 74) Chertin B., Puri P. Familial vesicoureteral reflux. J. Urol. 2003; 169: 1804-8.
(Ref. 75) Jacobson S. H., Hansson S., Jakobsson B. Vesico-ureteric reflux: occurrence and long-term risks. Acta Paediatr. 1999; 88: 22-30.
(Ref. 76) Gelfand M. J., Koch B. L., Cordero G. G., Salmanzadeh A., Gartside P. S. Vesicoureteral reflux: subpopulations of patients defined by clinical variables. Pediatr. Radiol. 2000; 30: 121-4.
(Ref. 77) Jakobsson B., Jacobson S. H., Hjalmas K. Vesico-ureteric reflux and other risk factors for renal damage: identification of high- and low-risk children. Acta Paediatr. 1999; 88: 31-9.
(Ref. 78) Gervaix A., Galetto-Lacour A., Gueron T., Vadas L., Zamora S., Suter S., Girardin E. Usefulness of procalcitonin and C-reactive protein rapid tests for the management of children with urinary tract infection. Pediatr. Infect. Dis. J. 2001, 20: 507-11.
(Ref. 79) Galetto-Lacour A., Zamora S. A., Gervaix A. Bedside procalcitonin and C-reactive protein tests in children with fever without localizing signs of infection seen in a referral centre. Pediatrics 2003; 112:1054-60.
(Ref. 80) Tuerlinckx D., Vander Borght T., Glupczynski Y., Galanti L., Roelants V., Krug B., de Bilderling G., Bodart E. Is procalcitonin a good marker of renal lesions in febrile urinary tract infection?Eur. J. Pediatr. 2005; 164: 651-2.
(Ref. 81) Prat C., Dominguez J., Rodrigo C., Gimenez M., Azuara M., Blanco S., Ausina V. Use of quantitative and semiquantitative procalcitonin measurements to identify children with sepsis and meningitis. Eur. J. Clin. Microbiol. Infect. Dis. 2004; 23: 136-8.
(Ref. 82) Dubos F., Moulin F., Raymond J., Gendrel D., Breart G., Chalumeau M. Distinction des meningites bactériennes et virales de l′enfant: affinement d′une règle de décision clinique. Arch Pediatr. 2007; 14: 434-8.
(Ref. 83) Chalumeau M., Chemaitilly W., Trivin C., Adan L., Breart G., Brauner R. Central precocious puberty in girls: an evidence-based diagnosis tree to predict central nervous System abnormalities. Pediatrics 2002; 109: 61-7.
(Ref. 84) Bigot S., Leblond P., Foucher C., Hue V., D'Herbomez M., Foulard M. Apport du dosage de la procalcitonine pour le diagnostic de pyélonéphrite aiguë de l′enfant. Arch Pediatr. 2005; 12: 1075-80.
(Ref. 85) Gurgoze M. K., Akarsu S., Yilmaz E., Gödekmerdan A, Akça Z, Ciftçi I., Aygün A. D. Proinflammatory cytokines and procalcitonin in children with acute pyelonephritis. Pediatr. Nephrol. 2005; 20: 1445-8.
(Ref. 86) Guven A. G., Kazdal H. Z., Koyun M., Aydn F., Gungor F., Akman S., Baysal Y. E. Accurate diagnosis of acute pyelonephritis: How helpful is procalcitonin? Nucl. Med. Commun. 2006; 27: 715-21.
(Ref. 87) Andreola B., Bressan S., Callegaro S., Liverani A., Plebani M., Da Dalt L. Procalcitonin and C-reactive protein as diagnostic markers of severe bacterial infections in febrile infants and children in the emergency department. Pediatr. Infect. Dis. J. 2007; 26: 672-7.
(Ref. 88) Pecile P., Miorin E., Romanello C., Falleti E., Valent F., Giacomuzzi F., Tenore A. Procalcitonin: a marker of severity of acute pyelonephritis among children. Pediatrics 2004; 114: e249-54.
(Ref. 89) Smolkin V., Koren A., Raz R., Colodner R., Sakran W., Halevy R. Procalcitonin as a marker of acute pyelonephritis in infants and children. Pediatr. Nephrol. 2002; 17: 409-12.
(Ref. 90) Benador N., Siegrist C. A., Gendrel D., Greder C., Benador D., Assicot M., Bohuon C., Girardin E. Procalcitonin is a marker of severity of renal lesions in pyelonephritis. Pediatrics 1998; 102: 1422-5.
(Ref. 91) Fernandez Lopez A., Luaces Cubells C., Garcia Garcia J. J., Fernandez Pou J. Procalcitonin in pediatric emergency departments for the early diagnosis of invasive bacterial infections in febrile infants: results of a multicenter study and utility of a rapid qualitative test for this marker. Pediatr. Infect. Dis. J. 2003; 22: 895-903.
(Ref. 92) Prat C., Dominguez J., Rodrigo C., Gimenez M., Azuara M., Jimenez O., Gali N., Ausina V. Elevated serum procalcitonin values correlate with renal scarring in children with urinary tract infection. Pediatr. Infect. Dis. J. 2003; 22: 438-42.
(Ref. 93) Deeks J. J., Higgins J. P. T., Altman D. G., editors. Analysing and presenting results. In: Alderson P, Green S, Higgins J, editors. Cochrane Reviewers' Handbook 4.2.2 [updated November 2004]; Section 8. Available at the website: http://www.cochrane.org/resources/handbook/hbook.htm.
(Ref. 94) Gajdos V., Benattar C., Guérin S., Trioche P., Perreaux F., Mollet A., Foix L′Hélias L., Labrune P. Intérêt de la procalcitonine et de la C-réactive protéine pour la prédiction des infections bactériennes sévères (IBS) chez les nourrissons fébriles âgés de moins de trois mois aux urgences. Congrés National de la Société de Pédiatrie, Lille, France, June 2004. Arch. Pediatr. 2004; 11: 728.
(Ref. 95) Korczowski B., Piasecka K., Duhl B., Bijos A. Procalcitonin and C-reactive protein as markers of bacteriemia in children hospitalized with pneumonia and acute pyelonephritis. Pediatrica Polaska 2004; 129: 205-10.
(Ref. 96) Fettich J., Colarinha P., Fischer S., Frokier J., Gordon I., Hahn K., Kabasakal L., Mann M., Mitjavila M., Olivier P., Piepsz A., Porn U., Roca I., Sixt R., van Velzen J. Guidelines for direct radionuclide cystography in children. Eur. J. Nucl. Med. Mol. Imaging 2003; 30: B39-44.
(Ref. 97) Wasson J. H., Sox H. C., Neff R. K., Goldman L. Clinical prediction rules. Applications and methodological standards. N. Engl. J. Med. 1985; 313: 793-9.
(Ref. 98) Meisner M. Procalcitonin, a new, innovative infection parameter, biochemical and clinical aspects. Stuttgart, Germany: Thieme, 2000.
(Ref. 99) Oostenbrink R., van der Heijden A. J., Moons K. G., Moll H. A.

Prediction of vesico-ureteric reflux in childhood urinary tract infection: a multivariate approach. Acta Paediatr. 2000; 89: 806-10.
(Ref. 100) Moses L., Littenberg B., Shapiro D. Combining independent studies of a diagnostic test into a summary ROC curve: data-analytical approaches and some additional consideration. Stat. Med. 1993; 12: 1293-316
(Ref. 101) Huguier M., Flahault A. Biostatistiques au quotidien. Paris, France: Elsevier, 2003
(Ref. 102) Falissard. Comprendre et utiliser les statistiques dans les sciences de la vie. 2005. 3rd edition. Ed Masson. Paris, France.
(Ref. 103) Bouyer J., Hemon D., Cordier S., Derrienic F., Stucker I., Stengel B., Clavel J. Epidemiologie. Principes et methodes. Paris: INSEM ed., 1993
(Ref. 104) Glas A. L., Prins M. H., Bonsel G. J., Bossuyt P. M. The diagnostic odds ratio: a single indicator of test performance. J. Clin. Epidemiol. 2003; 56: 1129-35
(Ref. 105) Higgins J. P., Thompson S. G., Deeks J. J., Altman D. G. Measuring inconsistency in meta-analyses. BMJ 2003; 327: 557-60.
(Ref. 106) Greenland S. Tests for interaction in epidemiologic studies: a review and study of power. Stat. Med. 1983; 2: 243-251
(Ref. 107) Fisher R. A. Statistical methods for research workers. 1934. Edition Oliver and Boyd, Edinburgh
(Ref. 108) Royston, Sauerbrei. Multivariable model building. A pragmatic approach to regression analysis based on fractional polynomials for modelling continuous variables. 2008. Ed: Wiley and Sons Ltd, Chichester, England
(Ref. 109) Hosmer, Lemeshow. Applied logistic regression. 2000 2nd edition. Ed: Wiley-Interscience Publication, New York.
(Ref. 110) Greenland S. Tests for interaction in epidemiologic studies: a review and study of power. Stat Med 1983; 2: 243-251.
(Ref. 111) Armitage, Barry, Matthews. Statistical methods in medical research. 4th edition. 2002. Ed: Blackwell science Ltd, Berlin, Germany

1-13. (canceled)

14. An in vitro method for predicting high-grade vesicoureteral reflux in a patient, comprising the following steps:
a) in vitro measurement of a serum concentration of procalcitonin in a biological sample from said patient;
b) assigning a procalcitonin value of 1 if the procalcitonin concentration is greater than or equal to a concentration of 1 ng/ml in said sample, or assigning a procalcitonin value of 0 if the procalcitonin concentration is not greater than or equal to a concentration of 1 ng/ml in said sample,
c) searching for dilation of at least one ureter, shown on a renal ultrasound image of said patient, assigning an ultrasound value of 1 in the event of dilation of at least one ureter or of an ultrasound value of 0 in the event of nondilation, and
d) summating said procalcitonin and ultrasound values obtained in steps b) and c), respectively.

15. The method as claimed in claim 14, wherein said summing step having a summation greater than or equal to 1 indicates that the patient is at high risk of vesicoureteral reflux, with a sensitivity of 86% for the prediction of high-grade vesicoureteral reflux.

16. The method as claimed in claim 14, wherein said summing step having a summation equal to 0 indicates that the patient has a low risk of high-grade vesicoureteral reflux, with a probability of misjudging a high-grade vesicoureteral reflux of less than 5%.

17. An in vitro method for predicting high-grade vesicoureteral reflux in a patient, comprising the following steps:
a) in vitro measuring of a serum concentration of procalcitonin in a biological sample from said patient,
b) searching for dilation of at least one ureter, shown on a renal ultrasound image of said patient, and assigning an ultrasound value of 1 in the event of dilation of at least one ureter or of an ultrasound value of 0 in the event of nondilation,
c) determining a reflux value Vr according to the following equation:
Vr=1/[1+exp(−a×ureteral dilation−b×[(Pct/100)^{(c)}+d]−e)]
in which a, b, c, d and e are decimal numbers resulting from a logistical regression equation which combines the ureteral dilation and the procalcitonin transformed into a fractional polynomial of degree 1, and
in which each value of a, b, c, d and e is independently included between −50 and 50, wherein a reflux value greater than a value included between 0 and 1 indicates that the patient is at high risk of high-grade vesicoureteral reflux.

18. The method for predicting high-grade vesicoureteral reflux in a patient, as claimed in claim 17, in which the values of a, b, c, d and e are −1.5 for a, −0.1 for b, −0.5 for c, −4.9 for d and −1.5 for e.

19. The method for predicting high-grade vesicoureteral reflux in a patient, as claimed in claim 17, in which a reflux value greater than or equal to a value of 0.072 indicates that the patient is at high risk of high-grade vesicoureteral reflux, with a sensitivity of 86% and a specificity of 47%.

20. The method for predicting high-grade vesicoureteral reflux in a patient, as claimed in claim 17, in which a reflux value less than a value of 0.072 indicates that the patient is at low risk of high-grade vesicoureteral reflux, with a probability of misjudging a high-grade vesicoureteral reflux of less than 5%.

21. The method as claimed in claim 17, in which the sample and the ultrasound image come from a patient with a first febrile urinary infection.

22. The method as claimed in claim 17, in which the patient is a child from 1 month to 4 years old.

23. The method as claimed in claim 17, in which the measurement of the procalcitonin concentration in the biological sample in step a) is carried out by means of a technique selected from the group consisting of an immunoassay, an immunoluminometric technique and an immunochromatographic technique.

24. The method as claimed in claim 17, in which the image obtained in step c) is an image obtained by means of a renal ultrasound medical imaging method.

25. A kit comprising:
(1) means for measuring the procalcitonin concentration in a biological sample from a patient, and
(2) a means for measuring the cross section of a ureter of a patient.

26. The kit as claimed in claim 25, in which the means for measuring the procalcitonin concentration comprises reagents for implementing a technique for measuring procalcitonin concentration, selected from the group consisting of an immunoassay, a Lumitest-PCT immunoluminometric technique, and a PCT-Q rapid semiquantitative immunochromatographic test (BRAHMS AG, Hennigsdorf).
